(19) United States
(12) Patent Application Publication
(10) Pub. No.: US 2024/0197868 A1
(43) Pub. Date: Jun. 20, 2024

(54) IMMUNOGENIC COMPOSITION CONTAINING AN ANTIGEN AND AN ADJUVANT COMPRISING AL-MOFS

(71) Applicants: UNIVERSITE DE VERSAILLES-SAINT-QUENTIN-EN-YVELINES, Versailles (FR); CENTRE NATIONAL DE LA RECHERCHE SCIENTIFIQUE, Paris (FR); BUREAU D’ÉTUDES BIOLOGIQUES SCIENTIFIQUES ET MÉDICALES, Les Septvallons (FR)

(72) Inventors: Jacques Henri Max COHEN, Reims (FR); Clémence SICARD, Versailles (FR); Effrosyni GKANIATSOU, Versailles (FR)

(73) Assignees: UNIVERSITE DE VERSAILLES-SAINT-QUENTIN-EN-YVELINES, Versailles (FR); CENTRE NATIONAL DE LA RECHERCHE SCIENTIFIQUE, Paris (FR); BUREAU D’ÉTUDES BIOLOGIQUES SCIENTIFIQUES ET MÉDICALES, Les Septvallons (FR)

(21) Appl. No.: 18/285,287

(22) PCT Filed: Apr. 1, 2022
(86) PCT No.: PCT/EP2022/058789

§ 371 Date: Oct. 2, 2023

(30) Foreign Application Priority Data
Apr. 2, 2021 (EP) 21305431.5

(51) Int. Cl. A61K 39/39; A61K 39/00; A61K 39/08; A61K 39/09; A61K 39/108; A61K 39/13; A61K 47/69; A61P 31/04; A61P 31/12; A61P 37/04

(52) U.S. Cl. CPC A61K 39/39; A61K 39/0258; A61K 39/08; A61K 39/092; A61K 39/13; A61K 47/6949; A61P 31/04; A61P 31/12; A61P 37/04; A61K 2039/521; A61K 2039/55505; A61K 2039/55511; A61K 2039/55544; A61K 2039/55561

(57) ABSTRACT

An immunogenic composition containing at least one antigen and at least one adjuvant including at least one Metal-Organic Framework including an inorganic part based on aluminum and an organic part based on polydentate ligand chosen from fumarate, muconate, mesaconate, oxalate, oxaloacetate, succinate, malate, citrate, aconitate, isophthalate, substituted isophthalate, 2,5-thiophenedicarboxylate, 2,5-furandicarboxylate, trimesate, trimellitate and pyromellitate, the antigen being immobilized at least within the Metal-Organic Framework.

This disclosure relates to the field of immunogenic compositions and in particular vaccine adjuvants.

More specifically, the instant invention relates to the use of specific aluminum metal-organic frameworks (Al-MOFs) systems, as an antigen delivery vehicle as well as an adjuvant to induce potent immune responses.

The field of vaccines is set apart from the rest of the pharmaceutical industry due to the specificities of the sector. Indeed, there is a much higher safety requirement for products administered to a healthy population, or even to the entire population, as well as a requirement for efficacy leading to the systematic verification of the batches and their release by laboratories of public checks, and not by the manufacturer itself.

Adjuvant formulations have been used for many years in vaccine compositions to enhance the immune response with the aim to confer long-term protection against targeted pathogens. Thus, adjuvants for vaccines are often essential for triggering an immune response and obtaining strong and lasting protective immunization. Adjuvants are also very useful to reduce the needed amount of a given antigen, while maintaining an efficient level of immune response of the vaccine. At last, it is also known that some adjuvants are only convenient for certain antigens, while others have a broader range of action and are effective in combination with antigens of different chemical natures and against different kinds of diseases.

Among the already available adjuvants, aluminum salts are the reference ones for non-living vaccines owing to their excellent inflammatory/immunostimulant ratio and their unique ability to boost immune responses of various antigens, despite intensive research for alternatives.

However, even if the risk to benefit ratio is excellent, these adjuvants are not resorptives, and are thus not degraded in vivo leaving indelible deposits. This is particularly unfortunate since aluminum salts are the best and almost the only efficiency/low local reaction compromise that can be used in humans.

Further, for such adjuvants, the antigens are adsorbed on the salts surface, which is not suitable for all antigens.

Other alternatives have already been developed (squalene, liposomes, etc.) but have so far not been as satisfactory as aluminum adjuvants. Therefore, for most inactivated vaccines, there is no practical alternative to the use of aluminum adjuvants.

Another concern when formulating adjuvants which are meant to be used by humans or animals, is that the preparation steps should be performed using products with low toxicity that are generally accepted by Health Agencies. For example, toxic solvents, such as DMF, should be avoided, and others, which have better acceptance from a pharmaceutical standpoint, should be preferred.

Therefore, there is still a need for new vaccine adjuvants as alternatives to the current aluminum adjuvants. In particular, there is a need for a novel aluminum-based material that will gradually disappear after processing, and thus for a novel aluminum-based material which is resorptive.

In other words, there is a need of an aluminum-based adjuvant able to degrade itself after fulfilling its role as adjuvant and as antigen's cargo.

There also remains a need for new formulations, such as adjuvant compositions, which are at least as effective, and even, more effective, in terms of immune response enhancement as the formulations that are available on the market, and thus may favor the presentation of the antigen.

There is also a need for adjuvant formulations, which allow an immobilization easy to implement for a wide range of antigens, and also for a combination of antigens and immune orienters.

In addition, an adjuvant to reduce the dose of the immunogen can be advantageous when the latter is expensive or complicated to produce on large scale. A better, more intense and above all more lasting antibody response is always desirable, for better protection and to limit the number of desirable boosters.

There is also a need for adjuvant formulations which are easy to manufacture, notably at industrial scale, involving raw materials, manufacturing intermediates and processes that are considered safe by most Health Authorities.

The present invention has for purpose to satisfy all or part of those needs.

The invention is thus directed to an immunogenic composition containing at least one antigen and at least one adjuvant with said adjuvant comprising at least one Metal-Organic Framework, MOF, comprising an inorganic part based on aluminum and an organic part based on at least one polydentate ligand, and said antigen being immobilized at least within said Metal-Organic Framework.

Therefore, according to one of its aspects, the invention is directed to an immunogenic composition containing at least one antigen and at least one adjuvant with said adjuvant comprising at least one Metal-Organic Framework, MOF, comprising an inorganic part based on aluminum and an organic part based on at least one polydentate ligand chosen from fumarate, muconate, mesaconate, oxalate, oxaloacetate, succinate, malate, citrate, aconitate, isophthalate, substituted isophthalate, 2,5-thiophenedicarboxylate, 2,5-furandicarboxylate, trimesate, trimellitate and pyromellitate, and said antigen being immobilized at least within said Metal-Organic Framework.

Preferably, the invention is directed to an immunogenic composition containing at least one antigen and at least one adjuvant with said adjuvant comprising at least one Metal-Organic Framework, MOF, comprising an inorganic part based on aluminum and an organic part which at least comprises a fumarate, and said antigen being immobilized at least within said Metal-Organic Framework.

As used herein, the term “immobilized” is intended to mean that the antigen is associated with the Metal-Organic Framework, and at least within the Metal-Organic Framework and the antigen is no longer in the fluid phase. The immobilization may occur by different ways. The immobilization may occur using a single step process, meaning the MOF formation and the immobilization is occurring simultaneously. The Metal-Organic Framework may entrap, by surrounding or encapsulating, the antigen. In some other conditions, the antigen may also be included into the pores of the Metal-Organic Framework, or the antigen may be adsorbed onto the external surface of the Metal-Organic Framework. The antigen can also be linked to the Metal-Organic Framework, notably by covalent bonds. It is understood that the antigens immobilization is not limited to these types of immobilization and can take place in different ways in the same Metal-Organic Framework.

The antigen may be immobilized within the MOF means that although not necessarily located in the pores of the MOF, the antigen is entrapped between MOF particles forming a non-soluble phase.

It is understood that all the antigens or some antigens are immobilized within said Metal-Organic Framework. Thus, according to one embodiment, all the antigens are immobilized within said Metal-Organic Framework. According to another embodiment, some antigens are immobilized within said Metal-Organic Framework and other antigens may be immobilized by said Metal-Organic Framework, for example at the surface of said Metal-Organic Framework.

Unexpectedly, the inventors discovered that specific coordination polymers called Metal-Organic Frameworks, MOFs, associated with aluminum, appear to be particularly efficient adjuvants, allowing to overcome the here-above detailed insufficiencies and thus, to achieve better potent immune responses.

Metal-Organic Frameworks (MOFs) are hybrid materials that have already demonstrated a strong potential for the vectorization and controlled release of pharmaceuticals, and which can be degraded in vivo. In particular, some MOF-based materials have been used as matrices for entrapping therein some antigens but never as the only adjuvants for the formulation of vaccines. Indeed, in terms of vaccine adjuvants, no antigens were immobilized within MOFs based on aluminum that were used as the single adjuvant molecule, to the knowledge of the inventors.

In particular, the present invention is based on the association of a coordination polymer (Metal-Organic Framework) based on aluminum (denoted Al-MOF) with any antigen (denoted Ag), in particular pro-antigen, biological or chemical molecule such as capable of directly or indirectly arousing in a living organism a specific immune response for prophylactic or therapeutic vaccine referred to herein as immunogen. For this, the immunogen is immobilized within an Al-MOF network in a single step process under physico-chemical conditions chosen to preserve the antigenic properties of the immunogen or induced by it.

Contrary to known aluminum adjuvants, aluminum-based MOFs according to the invention are resorptives, and allow the immobilization of any type of antigens. Further, the adjuvant according to the invention shows a better immune response than the known aluminum adjuvants.

Indeed, as shown in the following examples, aluminum-Metal-Organic Framework based adjuvant of the invention degrades while fulfilling its role unlike the reference product. Aluminum polydentate ligand MOFs preserve the adjuvant characteristics of aluminum, but with the advantage that the material will be gradually degraded into its chemical constituents, the exogenous organic ligand, and soluble Al³⁺ ions. The aluminum will therefore be dissolved, allowing its temporary presence at the injection site.

In addition, MOFs, as the ones considered according to the invention, are matrices wherein it is possible to immobilize a large amount of antigen with a very large presentation surface, and therefore that allow reducing the amounts of immunogen and adjuvant required.

WO 2021/097194 describes therapeutic agents encapsulated within a Metal-Organic Framework, notably based on Zinc. Such document does not describe aluminum-Metal-Organic Framework. Further, the inventors of the present application showed that such MOFs are not suitable for the immobilization of all antigens.

According to another of its aspect, the invention is directed to a Metal-Organic Framework comprising an inorganic part based on aluminum and an organic part based on at least one polydentate ligand chosen from fumarate, muconate, mesaconate, oxalate, oxaloacetate, succinate, malate, citrate, aconitate, isophthalate, substituted isophthalate, 2,5-thiophenedicarboxylate, 2,5-furandicarboxylate, trimesate, trimellitate and pyromellitate, for use to immobilize an antigen, in an immunogenic composition, and preferably in a vaccine adjuvant, said antigen being immobilized at least within said Metal-Organic Framework.

According to another of its aspect, the invention is directed to use of a Metal-Organic Framework comprising an inorganic part based on aluminum and an organic part based on polydentate ligand chosen from fumarate, muconate, mesaconate, oxalate, oxaloacetate, succinate, malate, citrate, aconitate, isophthalate, substituted isophthalate, 2,5-thiophenedicarboxylate, 2,5-furandicarboxylate, trimesate, trimellitate and pyromellitate, to immobilize an antigen, in an immunogenic composition, and preferably in a vaccine adjuvant, said antigen being immobilized at least within said Metal-Organic Framework.

In some preferred embodiments, such an immunogenic composition may be used as a vaccine composition. Preferably, the immunogenic composition, in particular the vaccine composition, is resorptive.

Furthermore, the invention proposes a simple and biologically compatible method to synthesize MOFs by a coordination reaction between aluminum compound and polydentate ligand in contact with the target antigen.

Thus, according to another of its aspect, the invention is directed to a process for preparing an immunogenic composition as defined above, comprising at least the step consisting to react at least one aluminum compound with at least one polycarboxylic acid chosen from fumaric acid, muconic acid, mesaconic acid, oxalic acid, oxaloacetic acid, succinic acid, malic acid, citric acid, aconitic acid, isophthalic acid, substituted isophthalic acid, 2,5-thiophenedicarboxylic acid, 2,5-furandicarboxylic acid, trimesic acid, trimellitic acid or pyromellitic acid and/or with at least one polycarboxylate chosen from fumarate, muconate, mesaconate, oxalate, oxaloacetate, succinate, malate, citrate, aconitate, isophthalate, substituted isophthalate, 2,5-thiophenedicarboxylate, 2,5-furandicarboxylate, trimesate, trimellitate and pyromellitate, in the presence of at least one antigen, for forming at least one Al-polycarboxylate Metal-Organic Framework immobilizing said antigen.

Since the synthesis takes place advantageously under mild and sustainable conditions, its scaling up presents no particular difficulty. In addition, the production cost of MOF remains advantageously low (synthetic precursors at low cost, process in water and at room temperature).

Thus, the inventors have specifically also developed a process for manufacturing a MOF based on polydentate ligand as described above and aluminum under physicochemical conditions, which makes it possible to immobilize biological entities without denaturing them.

Unexpectedly, the presentation surface of the antigen immobilized in the MOF structure obtained according to the invention, is advantageously considerably greater than that of quasi-macroscopic precipitates of protein and aluminum salts currently used. Further, as shown in the following examples, the immobilization capacity of Al-MOF was 100% wt., immobilizing the totality of the added antigen. This possibility makes possible to immobilize a large quantity of antigen with a very large presentation surface.

As disclosed in the following examples, the gradual degradation of Al-MOF under physiological conditions has been also proved in vivo.

### Immunogenic Composition

The disclosure relates to an immunogenic composition. In some preferred embodiments, such an immunogenic composition may be used as a vaccine composition.

As used herein, the term “vaccine” is intended to mean a direct or indirect immunogenic composition, which is administered to a subject to induce an immune response with the intent to protect or treat the subject from an illness caused by the pathogen agent.

A vaccine composition is thus a composition which is used to elicit a protective immune response to a given antigen. A vaccine is usually used as a prevention tool, but may also, in certain cases, be used as a treatment.

Among vaccines, mention be made of prophylactic vaccines and therapeutic vaccines. Prophylactic vaccines are vaccines administrated for the prevention of infectious diseases, and which immunize a subject before exposition to the pathogens responsible of these diseases. Therapeutic vaccines are vaccines intended to stimulate the immune system by inducing it to reject for example cancer cells or to recreate a specific immune response. Contrary to prophylactic vaccines, which are essentially preventives, therapeutic vaccines are mainly administrated as a treatment to subjects already suffering from specific diseases, such as cancer or AIDS.

The immunogenic composition, or vaccine composition, according to the present disclosure includes an antigen for inducing immunity and an aluminum based Metal-Organic Framework (MOF). The MOF mainly functions as an adjuvant in the composition, together with its role in immobilization and preservation of the antigen.

### Adjuvant

According to the invention, the adjuvant is a vaccine adjuvant.

Within the disclosure, the term “adjuvant” or “adjuvant effect” is used to qualify a compound or composition which, when added to an antigen-containing immunogenic composition, or an antigen-containing vaccine composition, efficiently triggers or enhances an immune response to the antigen by, e.g. enhancing antigen presentation to antigen-specific immune cells and/or by activating these cells with the aim to confer long-term protection against targeted pathogens.

Preferably, the adjuvant according to the invention is resorptive. Resorptive means that the immunogen is absorbable, and thus disappearing or vanishing with time from the injection site.

In particular, less than 40% by weight of the injected aluminum remains at the injection site after 1 month, preferably less than 30% by weight, and preferably less than 25% by weight.

More particularly, less than 30% by weight of the injected aluminum remains at the injection site after 2 months, preferably less than 25% by weight, and preferably less than 10% by weight.

More particularly, less than 20% by weight of the injected aluminum remains at the injection site after 3 months, preferably less than 15% by weight, and more preferably less than 6% by weight.

The adjuvant according to the invention comprises at least one Metal-Organic Framework comprising an inorganic part based on aluminum and an organic part based on polydentate ligand.

According to a particular embodiment, an immunogenic composition according to the present invention may comprise other adjuvants than the adjuvant comprising at least one Metal-Organic Framework.

### Metal-Organic Framework (MOFs)

A Metal-Organic Framework (MOF), also named Coordination Polymer, is a hybrid solid containing inorganic units and organic ligands. The MOFs typically form a structure, preferably a porous structure, by combination of a metal and a polydentate ligand.

According to the invention, the MOF is configured to decompose in vivo.

Any kinds of MOFs comprising an inorganic part based on aluminum and an organic part based on at least one polydentate ligand as described above, can be used in the immunogenic composition.

Appropriately combining the type and coordination number of the aluminum ion with the type and topology of the polydentate ligand leads to a MOF with a desired structure.

The MOF can be crystalline or amorphous.

Preferably, the Metal-Organic Framework is crystallized.

Preferably, the Metal-Organic Framework is porous.

The combination of the aluminum compound and the ligand forming the MOF can be appropriately determined according to the expected function and the desired pore size.

The MOF of the invention may thus comprise pores, in particular micropores and/or mesopores. Micropores are defined as pores having a diameter of less than 2 nm and mesopores are defined by a diameter in the range of 2 to 50 nm, in each case corresponding to the definition given in IUPAC or in Pure Applied Chem. 57 (1985), pages 603-619.

The presence of micropores and/or mesopores can be checked by means of sorption measurements.

The MOF can be present in powder form or as agglomerate.

According to a preferred embodiment, the MOF according to the invention is not implemented in a separate vehicle, and preferably is not implemented in a yeast.

### Inorganic Part Based on Aluminum

This is preferably an aluminum compound chosen from aluminum salt, aluminum oxide, aluminum hydroxide, and aluminum alkoxide, or a mixture thereof.

Preferably, the aluminum compound is chosen from aluminum salt, aluminum oxide and aluminum hydroxide, or a mixture thereof.

Aluminum salts include inorganic aluminum salts and organic aluminum salts.

Inorganic aluminum salts may be chosen from aluminum nitrate, aluminum sulfate, aluminum phosphate, aluminum carbonate, aluminum halides, and aluminum perchlorate.

Aluminum halides may be aluminum chlorides, aluminum bromides, aluminum fluorides or aluminum iodides.

Organic aluminum salts may be chosen from aluminum oxalate, aluminum acetate, aluminum stearate, aluminum lactate, aluminum laurate and aluminum citrate.

Aluminum acetate may be basic aluminum monoacetate, basic aluminum diacetate, or neutral aluminum triacetate.

Aluminum alkoxide notably includes aluminum isopropoxide, aluminum ethoxide and aluminum butoxide.

It is clear that it is possible to envisage using a blend of the various aluminum compounds mentioned above.

Particular preference is given to aluminum sulfate, either as anhydrous or hydrate, in particular in the form of its octadecahydrate or tetradecahydrate.

As at least one aluminum compound, it is also possible to use an aluminate.

Such as an alkali metal aluminate may in particular be NaAlO₂. Since this has basic properties, the presence of a base in the reaction can be dismissed. However, it is also possible to use an additional base.

Preferably, the inorganic part based on aluminum is formed from aluminum sulfate.

According to one embodiment, the mass ratio antigen/aluminum varies from 10⁻⁵ to 1, and preferably from 10⁻² to 10⁻¹, when the antigen is tetanus toxoid.

It is understood that such ratio antigen/aluminum will depend on the selected antigen.

The MOF of the invention comprises at least aluminum ion as metal ion.

In one embodiment, aluminum ion is the only one metal ion in the MOF framework.

In another embodiment, more than one metal ion is present in the MOF.

These one or more metal ions other than aluminum can be located in the pores of the MOF or participate in the formation of the lattice of the framework. In the latter case, the at least one polydentate organic compound would likewise be bound to such a metal ion.

If more than one metal ion is comprised in the MOF, these can be present in a stoichiometric or nonstoichiometric amount.

Preferably, the MOF has only one metal ion, and more preferably aluminum.

### Organic Part Based on Polydentate Ligand

As used herein, a “polydentate ligand” means a ligand that can form two or more coordination bonds, and is understood as defined by IUPAC.

Examples of organic polydentate ligands include the ligands listed in WO2010/075610.

Preferably, the used ligand is nontoxic.

The polydentate ligand in the MOF typically is an organic ligand, examples of which include carboxylate anions and heterocyclic compounds. Examples of the carboxylic acid anions include dicarboxylic acid anions and tricarboxylic acid anions.

These ones or more further at least polydentate organic compounds are for example derived from a dicarboxylic, tricarboxylic or tetracarboxylic acid. Other at least polydentate organic compounds can also participate in the formation of a framework. However, it is likewise possible for organic compounds which are not at least polydentate also to be comprised in a framework. These can be derived, for example, from a monocarboxylic acid.

For the purposes of the present invention, the term “derived” means that the dicarboxylic, tricarboxylic or tetracarboxylic acid can be present in partially deprotonated or completely deprotonated form in the framework. Furthermore, the dicarboxylic, tricarboxylic or tetracarboxylic acid can comprise a substituent or a plurality of independent substituents. Non limitative examples of such substituents are —OH, —NH₂, —OCH₃, —CH₃, —NH(CH₃), —N(CH₃)₂, —CN and halides. Furthermore, the term “derived” as used for the purposes of the present invention means that the dicarboxylic, tricarboxylic or tetracarboxylic acid can also be present in the form of the corresponding sulfur analogues. Sulfur analogues are the functional groups —C(═O)SH and the tautomer thereof and C(═S)SH, which can be used instead of one or more carboxylic acid groups. Furthermore, the term “derived” as used for the purposes of the present invention means that one or more carboxylic acid functions can be replaced by a sulfonic acid group (—SO₃H). In addition, a sulfonic acid group can likewise be present in addition to the 2, 3 or 4 carboxylic acid functions. Furthermore, the term “derived” as used for the purposes of the present invention means that one or more carboxylic acid functions can be in the form of salts, for example, carboxylate sodium salt or carboxylate potassium salt.

The dicarboxylic, tricarboxylic or tetracarboxylic acid may have, in addition to the above-mentioned functional groups, an organic skeleton or an organic compound to which these functional groups are bound. Here, the above-mentioned functional groups can in principle be bound to any suitable organic compound as long as it is ensured that the organic compound bearing these functional groups is suitable for forming the coordinate bond for producing the framework.

Organic compounds may be derived from a saturated or unsaturated aliphatic compound or an aromatic compound or a both aliphatic and aromatic compound.

An aliphatic compound or an aliphatic part of the both aliphatic and aromatic compound can be linear and/or branched and/or cyclic, with a plurality of rings per compound also being possible. An aliphatic compound or the aliphatic part of the both aliphatic and aromatic compound for example comprises from 1 to 18, more preferably from 1 to 14, more preferably from 1 to 13, more preferably from 1 to 12, more preferably from 1 to 11 and particularly preferably from 1 to 10, carbon atoms, for example 1, 2, 3, 4, 5, 6, 7, 8, 9 or 10 carbon atoms. In particular, it can be methane, adamantane, acetylene, ethylene or butadiene.

An aromatic compound or an aromatic part of both aromatic and aliphatic compound can have one or more rings, for example two, three, four or five rings, with the rings being able to be present separately from one another and/or at least two rings being able to be present in condensed form. An aromatic compound or an aromatic part of the both aliphatic and aromatic compound particularly has one, two or three rings, with one or two rings being particularly preferred. Each ring of said compound can independently comprise at least one heteroatom such as N, O, S, B, P, Si, for example N, O and/or S. An aromatic compound or an aromatic part of the both aromatic and aliphatic compound may comprise one or two C6 rings, with the two rings being present either separately or in condensed form. Particular mention may be made of benzene, naphthalene and/or biphenyl and/or bipyridyl and/or pyridyl as aromatic compounds.

A polydentate organic compound is for example an aliphatic or aromatic, acyclic or cyclic hydrocarbon having from 1 to 18, preferably from 1 to 10 and in particular 6, carbon atoms and having exclusively 2, 3 or 4 carboxyl groups as functional groups.

For example, a polydentate organic compound is derived from a dicarboxylic acid such as fumaric acid, oxalic acid, succinic acid, malic acid, aspartic acid, glutamic acid, glutaric acid, tartaric acid, 1,4-butanedicarboxylic acid, 1,4-butenedicarboxylic acid, 4-oxopyran-2,6-dicarboxylic acid, 1,6-hexanedicarboxylic acid, decanedicarboxylic acid, 1,8-heptadecanedicarboxylic acid, 1,9-heptadecanedicarboxylic acid, heptadecanedicarboxylic acid, acetylenedicarboxylic acid, 1,2-benzenedicarboxylic acid, 1,3-benzenedicarboxylic acid, 2,3-pyridinedicarboxylic acid, pyridine-2,3-dicarboxylic acid, 1,3-butadiene-1,4-dicarboxylic acid, 1,4-benzenedicarboxylic acid, p-benzenedicarboxylic acid, imidazole-2,4-dicarboxylic acid, 2-methylquinoline-3,4-dicarboxylic acid, quinoline-2,4-dicarboxylic acid, quinoxaline-2,3-dicarboxylic acid, 6-chloroquinoxaline-2,3-dicarboxylic acid, 4,4′-diaminophenylmethane-3,3′-dicarboxylic acid, quinoline-3,4-dicarboxylic acid, 7-chloro-4-hydroxyquinoline-2,8-dicarboxylic acid, di imidedicarboxylic acid, pyridine-2,6-dicarboxylic acid, 2-methylimidazole-4,5-dicarboxylic acid, thiophene-3,4-dicarboxylic acid, 2-isopropylimidazole-4,5-dicarboxylic acid, tetrahydropyran-4,4-dicarboxylic acid, perylene-3,9-dicarboxylic acid, perylenedicarboxylic acid, 3,6-dioxaoctanedicarboxylic acid, 3,5-cyclohexadiene-1,2-dicarboxylic acid, octanedicarboxylic acid, pentane-3,3-carboxylic acid, 4,4′-diamino-1,1′-biphenyl-3,3′-dicarboxylic acid, 4,4′-diaminobiphenyl-3,3′-dicarboxylic acid, benzidine-3,3′-dicarboxylic acid, 1,4-bis(phenylamino)benzene-2,5-dicarboxylic acid, 1,1′-binaphthyldicarboxylic acid, 7-chloro-8-methylquinoline-2,3-dicarboxylic acid, 1-anilinoanthraquinone-2,4′-dicarboxylic acid, polytetrahydrofuran 250-dicarboxylic acid, 1,4-bis(carboxymethyl)piperazine-2,3-dicarboxylic acid, 7-chloroquinoline-3,8-dicarboxylic acid, 1-(4-carboxy)phenyl-3-(4-chloro)phenylpyrazoline-4,5-dicarboxylic acid, 1,4,5,6,7,7-hexachloro-5-norbornene-2,3-dicarboxylic acid, phenylindanedicarboxylic acid, 1,3-dibenzyl-2-oxoimidazolidine-4,5-dicarboxylic acid, 1,4-cyclohexanedicarboxylic acid, naphthalene-1,8-dicarboxylic acid, 2-benzoylbenzene-1,3-dicarboxylic acid, 1,3-dibenzyl-2-oxoimidazolidine-4,5-cis-dicarboxylic acid, 2,2′-biquinoline-4,4′-dicarboxylic acid, pyridine-3,4-dicarboxylic acid, 3,6,9-trioxaundecanedicarboxylic acid, hydroxybenzophenonedicarboxylic acid, the commercial compounds Pluriol E 200-dicarboxylic acid, Pluriol E 300-dicarboxylic acid, Pluriol E 400-dicarboxylic acid, and Pluriol E 600-dicarboxylic acid, pyrazole-3,4-dicarboxylic acid, 2,3-pyrazincdicarboxylic acid, 5,6-dimethyl-2,3-pyrazinedicarboxylic acid, bis(4-aminophenyl)ether diimide-dicarboxylic acid, 4,4′-diaminodiphenylmethane diimide-dicarboxylic acid, bis(4-aminophenyl)sulfone diimide-dicarboxylic acid, 1,4-naphthalenedicarboxylic acid, 2,6-naphthalene-dicarboxylic acid, 1,3-adamantanedicarboxylic acid, 1,8-naphthalenedicarboxylic acid, 2,3-naphthalencdicarboxylic acid, 8-methoxy-2,3-naphthalenedicarboxylic acid, 8-nitro-2,3-naphthalenecarboxylic acid, 8-sulfo-2,3-naphthalenedicarboxylic acid, anthracene-2,3-dicarboxylic acid, 2′,3′-diphenyl-p-terphenyl-4,4″-dicarboxylic acid, (diphenyl ether)-4,4′-dicarboxylic acid, imidazole-4,5-dicarboxylic acid, 4(1H)-oxothiochromene-2,8-dicarboxylic acid, 5-tert-butyl-1,3-benzenedicarboxylic acid, 7,8-quinolinedicarboxylic acid, 4,5-imidazoledicarboxylic acid, 4-cyclohexene-1,2-dicarboxylic acid, hexatriacontanedicarboxylic acid, tetradecanedicarboxylic acid, 1,7-heptanedicarboxylic acid, 5-hydroxy-1,3-benzenedicarboxylic acid, 2,5-dihydroxy-1,4-benzenedicarboxylic acid, pyrazine-2,3-dicarboxylic acid, furan-2,5-dicarboxylic acid, 1-nonene-6,9-dicarboxylic acid, cicosenedicarboxylic acid, 4,4′-dihydroxy-diphenylmethane-3,3′-dicarboxylic acid, 1-amino-4-methyl-9,10-dioxo-9,10-dihydroanthracene-2,3-dicarboxylic acid, 2,5-pyridinedicarboxylic acid, cyclohexene-2,3-dicarboxylic acid, 2,9-dichlorofluorubin-4,11-dicarboxylic acid, 7-chloro-3-methylquinoline-6,8-dicarboxylic acid, 2,4-dichlorobenzophenone-2′,5′-dicarboxylic acid, 1,3-benzenedicarboxylic acid, 2,6-pyridinedicarboxylic acid, 1-methylpyrrol-3,4-dicarboxylic acid, 1-benzyl-1H-pyrrol-3,4-dicarboxylic acid, anthraquinone-1,5-dicarboxylic acid, 3,5-pyrazoledicarboxylic acid, 2-nitrobenzene-1,4-dicarboxylic acid, heptane-1,7-dicarboxylic acid, cyclobutane-1,1-dicarboxylic acid, 1,14-tetradecanedicarboxylic acid, 5,6-dehydronorbornane-2,3-dicarboxylic acid, 5-ethyl-2,3-pyridinedicarboxylic acid or camphordicarboxylic acid.

A polydentate organic compound may be for example one of the dicarboxylic acids mentioned above by way of example as such.

For example, a polydentate organic compound can be derived from a tricarboxylic acid such as 2-Hydroxy-1,2,3-propanetricarboxylic acid, 7-chloro-2,3,8-quinolinetricarboxylic acid, 1,2,3-, 1,2,4-benzenetricarboxylic acid, 1,2,4-butanetricarboxylic acid, 2-phosphono-1,2,4-butanetricarboxylic acid, 1,3,5-benzenetricarboxylic acid, 1-hydroxy-1,2,3-propanetricarboxylic acid, 4,5-dihydroxy-4,5-dioxo-1H-pyrrolo[2,3-F]quinoline-2,7,9-tricarboxylic acid, 5-acetyl-3-amino-6-methylbenzene-1,2,4-tricarboxylic acid, 3-amino-5-benzoyl-6-methylbenzene-1,2,4-tricarboxylic acid, 1,2,3-propanetricarboxylic acid or aurintricarboxylic acid.

A polydentate organic compound may be for example one of the tricarboxylic acids mentioned above by way of example as such.

Examples of a polydentate organic compound which is derived from a tetracarboxylic acid are 1,1-Dioxidoperylo[1,12-BCD]thiophene-3,4,9,10-tetracarboxylic acid, perylenetetracarboxylic acids such as perylene-3,4,9,10-tetracarboxylic acid or (perylene-1,12-sulfone)-3,4,9,10-tetracarboxylic acid, butanetetracarboxylic acids such as 1,2,3,4-butanetetracarboxylic acid or meso-1,2,3,4-butanetetracarboxylic acid, decane-2,4,6,8-tetracarboxylic acid, 1,4,7,10,13,16-hexaoxacyclooctadecane-2,3,11,12-tetracarboxylic acid, 1,2,4,5-benzenetetracarboxylic acid, 1,2,11,12-dodecanetetracarboxylic acid, 1,2,5,6-hexanetetracarboxylic acid, 1,2,7,8-octanetetracarboxylic acid, 1,4,5,8-naphthalenetetracarboxylic acid, 1,2,9,10-decanetetracarboxylic acid, benzophenonetetracarboxylic acid, 3,3′,4,4′-benzophenonetetracarboxylic acid, tetrahydrofurantetracarboxylic acid or cyclopentantetracarboxylic acids such as cyclopentane-1,2,3,4-tetracarboxylic acid.

A polydentate organic compound may be for example one of the tetracarboxylic acids mentioned above by way of example as such.

A polydentate organic compound may be for example chosen from optionally at least monosubstituted aromatic dicarboxylic, tricarboxylic or tetracarboxylic acids having one, two, three, four or more rings, where each of the rings can comprise at least one heteroatom, in which case two or more rings can comprise identical or different heteroatoms. A polydentate organic compound may be for example chosen from monocyclic dicarboxylic acids, monocyclic tricarboxylic acids, monocyclic tetracarboxylic acids, bicyclic dicarboxylic acids, bicyclic tricarboxylic acids, bicyclic tetracarboxylic acids, tricyclic dicarboxylic acids, tricyclic tricarboxylic acids, tricyclic tetracarboxylic acids, tetracyclic dicarboxylic acids, tetracyclic tricarboxylic acids and/or tetracyclic tetracarboxylic acids. Suitable heteroatoms are, for example, N, O, S, B, P, and in particular are N, S and/or O. A suitable substituent here is, inter alia, —OH, a nitro group, an amino group or an alkyl or alkoxy group.

A polydentate organic compound may be for example chosen from acetylenedicarboxylic acid (ADC), camphordicarboxylic acid, fumaric acid, succinic acid, malic acid, aspartic acid, glutamic acid, glutaric acid, benzenedicarboxylic acids, naphthalenedicarboxylic acids, acids biphenyldicarboxylic such as 4,4′-biphenyldicarboxylic acid (BPDC), pyrazinedicarboxylic acids such as 2,5-pyrazinedicarboxylic acid, bipyridinedicarboxylic acids such as 2,2′-bipyridinedicarboxylic acids such as 2,2′-bipyridine-5,5′-dicarboxylic acid, benzenetricarboxylic acids such as 1,2,3-, 1,2,4-benzenetricarboxylic acid or 1,3,5-benzenetricarboxylic acid (BTC), benzenetetracarboxylic acid, adamantanetetracarboxylic acid (ATC), adamantanedibenzoate (ADB) benzenetribenzoate (BTB), methanetetrabenzoate (MTB), adamantanetetrabenzoate or dihydroxyterephthalic acids such as 2,5-dihydroxyterephthalic acid (DHBDC).

A polydentate organic compound may be for example chosen from phthalic acid, isophthalic acid, terephthalic acid, 2,6-naphthalenedicarboxylic acid, 1,4-naphthalenedicarboxylic acid, 1,5-naphthalenedicarboxylic acid, 1,2,3-benzenetricarboxylic acid, 1,2,4-benzenetricarboxylic acid, 1,3,5-benzenetricarboxylic acid or 1,2,4,5-benzenetetracarboxylic acid.

Specific examples include anions of fumaric acid, citric acid, malic acid, terephthalic acid, isophthalic acid, succinic acid, aspartic acid, glutamic acid, glutaric acid, trimesic acid, and derivatives thereof. Examples of the heterocyclic compound include bipyridine, imidazole, adenine, and derivatives thereof. Alternatively, the ligand may be an amine compound, a sulfonate anion, or a phosphate anion.

Examples of polydentate ligand includes anions of 2,3-pyrazinedicarboxylic acid (pzdc); pyrazine; trimesic acid (BTC); terephthalic acid (BDC); 1,4-diazabicyclo[2,2,2]octane (dabco); imidazole; 1,3,5-benzenetricarboxylic acid; citric acid; malic acid; isophthalic acid; 2,5-dihydroxyterephthalic acid (HBDC); 4,4′-oxobisbenzoic acid (OBA); 1,3,5-tri(4′-carboxy-4,4′-biphenyl)benzene (BTB); 4,4′-4″-benzene-1,3,5-triyl-tri-biphenylcarboxylic acid (BBC); azelaic acid; zoledronic acid; o-bromoterephthalic acid (o-Br-BDC); 2-aminoterephthalic acid (H₂N-BDC); [C₃H₇O]₂-BDC; [C₅H₁₁O]₂-BDC; [C₂H₄]-BDC; 1,4-naphthalenedicarboxylic acid (1,4-NDC); 2,6-naphthalenedicarboxylic acid (2,6-NDC); 4,4′-biphenyldicarboxylic acid (BPDC); tetrahydropyrene-2,7-dicarboxylic acid (HPDC); pyrene dicarboxylic acid (PDC); terphenyl dicarboxylic acid (TPDC); formic acid; m-BDC; BzPDC; 5,5′-(9,10-anthracenediyl) diisophosphate; 1,1′-binaphthyl-4,4′-dicarboxylic acid (BNDC); 4,4′-biphenyldicarboxylic acid (BPDC); dibenzyl phosphate (DBP); 1,3,5,7-adamantanetetracarboxylic acid (ATC); acetylenedicarboxylic acid adamantanetetrabenzoic (ADC); acid (ATB); methanetetrabenzoic acid (MTB); oxalic acid; 1,4-diphenyl diacrylic acid (PDAC); 4,4′-stilbene dicarboxylic acid (SDBC); 1,3,5-tri(4′-carboxy-4,4′-biphenyl)benzene (BTB); 4,4′,4″-[benzene-1,3,5-triyl-tris(ethyne-2,1-diyl)] tribenzoic acid (BTE); 1,2,4,5-Tetrakis(4-carboxyphenyl)benzene (TCPB); 1,4-dicarboxylbenzene-2,3-dithiolate (DCBDT); caustic acid (3,4,5-trihydroxybenzoic acid).

For example, a polydentate organic compound may be derived from a dicarboxylic acid such as fumaric acid, malic acid, aspartic acid, glutamic acid or glutaric acid.

A MOF may further contain at least one monodentate ligand.

According to the invention, the polydentate ligand is chosen from fumarate, muconate, mesaconate, oxalate, oxaloacetate, succinate, malate, citrate, aconitate, isophthalate, substituted isophthalate, 2,5-thiophenedicarboxylate, 2,5-furandicarboxylate, trimesate, trimellitate and pyromellitate, or derived from fumaric acid, muconic acid, mesaconic acid, oxalic acid, oxaloacetic acid, succinic acid, malic acid, citric acid, aconitic acid, isophthalic acid, substituted isophthalic acid, 2,5-thiophenedicarboxylic acid, 2,5-furandicarboxylic acid, trimesic acid, trimellitic acid or pyromellitic acid.

In particular, the polydentate ligand is chosen from fumarate, muconate, mesaconate, succinate, malate, isophthalate, substituted isophthalate, 2,5-thiophenedicarboxylate, 2,5-furandicarboxylate, trimesate, trimellitate and pyromellitate, or derived from fumaric acid, muconic acid, mesaconic acid, succinic acid, malic acid, isophthalic acid, substituted isophthalic acid, 2,5-thiophenedicarboxylic acid, 2,5-furandicarboxylic acid, trimesic acid, trimellitic acid or pyromellitic acid.

More particularly, the polydentate ligand is chosen from fumarate, muconate, isophthalate, 2,5-thiophenedicarboxylate, 2,5-furandicarboxylate and trimesate, or derived from fumaric acid, muconic acid, isophthalic acid, 2,5-thiophenedicarboxylic acid, 2,5-furandicarboxylic acid or trimesic acid.

In particular, the polydentate ligand is chosen from fumarate, muconate, 2,5-furandicarboxylate, trimesate and pyromellitate, or derived from fumaric acid, muconic acid, 2,5-furandicarboxylic acid, trimesic acid or pyromellitic acid.

Preferably, the polydentate ligand is chosen from fumarate, muconate, 2,5-thiophenedicarboxylate, 2,5-furandicarboxylate and trimesate, or derived from fumaric acid, muconic acid, 2,5-thiophenedicarboxylic acid, 2,5-furandicarboxylic acid or trimesic acid.

Preferably, the polydentate ligand is chosen from fumarate, muconate, trimesate and pyromellitate, or derived from fumaric acid, muconic acid, 2 acid, trimesic acid or pyromellitic acid.

More preferably, the polydentate ligand is chosen from fumarate, muconate, and trimesate, or derived from fumaric acid, muconic acid or trimesic acid.

More preferably, the used ligand is fumarate, or derived from fumaric acid.

### MOF Structures

Depending on the organic ligand and/or the synthetic conditions, different structures can be obtained, thus numerous Al-MOF structures have been reported. Some examples are described below.

In particular, as Al-MOFs with dicarboxylic acid ligands, may be cited Al-MOFs with fumarate ligands, Al-MOFs with muconate ligands, Al-MOFs with mesaconate ligands, Al-MOFs with oxalate ligands, Al-MOFs with oxaloacetate ligands, Al-MOFs with succinate ligands, Al-MOFs with malate ligands, Al-MOFs with isophthalate ligands (1,3-benzenedicarboxylate), Al-MOFs with substituted isophthalate ligands, Al-MOFs with 2,5-thiophenedicarboxylate ligands, and Al-MOFs with 2,5-furandicarboxylate ligands.

Typical examples of dicarboxylic Al-MOF structures are the isostructural (similar topologies but with different organic ligands) series of MIL-53(Al) (MIL=“Matériaux Institut Lavoisier de Versailles”). MIL-53(Al) structures are composed by 1D AlO₄(OH)₂ chains of corner sharing Al(III) octahedral linked together by linear dicarboxylates (fumarate, muconate, etc..). The ligand can also be substituted with functional groups leading to MIL-53(Al) structures with reduced porosity as pending functional groups are present in the MOF channels.

When the dicarboxylates are fumarate ligands (derived from fumaric acid), the structure is Al-fumarate (or MIL-53(Al)-FA or Basolite A520), a microporous structure with 1D channels of 5.7×6.0 Å² free aperture.

When the dicarboxylates are muconate ligands (derived from muconic acid), the structure can be MIL-53(Al)-muc, a microporous structure with 1D channels of 9.0 Å free aperture.

Another Al-MOF structure is obtained when isophthalate ligands (1,3-benzenedicarboxylate) (derived from isophthalic acid) are connected with helical chains of cis-corner sharing AlO₆ octahedral. For example, CAU-10-H (CAU=Christian-Albrechts-Universitat), shows a 3D microporous structure with square shaped one dimensional channels of 3.6 Å² free aperture.

Alternatively, with 2,5-thiophenedicarboxylate, the CAU-23 structure is obtained with consecutive trans- and cis-corner-sharing A16 polyhedra resulting in square channel micropores of 7.6 Å free aperture.

A typical example of MOF obtained with the 2,5-furandicarboxylate ligand is the MIL-160(Al). MIL-160(Al) results from the connection of chains of AlO₄(OH)₂ octahedra with 2,5-furandicarboxylate ligands. This leads to a 3D structure with square-shaped sinusoidal 1D channels of approximately 5-6 Å in diameter.

As Al-MOFs with tricarboxylic acid ligands, may be cited Al-MOFs with trimesate ligands, Al-MOFs with trimellitate (1,2,4 benzene tricarboxylate) ligands, Al-MOFs with citrate ligands and Al-MOFs with aconitate ligands.

Based on the same building units, i.e. aluminum and trimesate (1,3,5-benzene tricarboxylate), different 3D frameworks can be obtained, such as MIL-96(Al), MIL-100(Al) and MIL-110(Al). MIL-96(Al) results from the assembly of aluminum trimers coordinated to trimesate ligands, and connected to an additional hexagonal 18-membered ring subunit built by—chains of aluminum octahedra. The microporosity of MIL-96(Al) consists of three types of cavities: a spherical cage with a cavity-free diameter of about 11 Å, an elongated cavity with dimensions of 9.5×12.6×11.3 Å and a narrow cavity with dimensions of 3.6×4.5 Å. MIL-100(Al) results from the connection of trimesate ligands and Al(III)trimers, leading to a mesoporous structure, with two kinds of cavities of different diameter (24 and 29 Å), accessible by microporous windows (5.2 and 8.8 Å). MIL-110(Al) has a three-dimensional framework composed of 8 aluminum octahedra linked through trimesate ligands to form a microporous structure, with hexagonal channels of 16 Å wide.

As Al-MOFs with tetracarboxylic acid ligands may be cited Al-MOFs with pyromellitate (1,2,4,5-benzene tetracarboxylate) ligands.

Different frameworks can be obtained, such as MIL-118(Al) or MIL-120(Al). MIL-118(Al) consists of infinite chains of trans-connected aluminum-centered octahedra linked to each other through the pyromellitate ligand. The framework can exhibit three different phases depending on the hydration/drying state. MIL-120(Al) consists of infinite chains of aluminum centers in octahedral coordination connected to each other through the pyromellitate ligand, resulting in the formation of channels of 5.4×4.7 Å².

Preferably, the polycarboxylate comprises fumarate.

The MOF may contain two or more types of ligands.

Only one type of MOF may be used, or two or more types thereof may be used in combination.

The MOF can be surface-modified with a polymer or other modifiers.

The content of the MOF in the immunogenic composition is, for example, in the range of 90 to 99.9 mass %, preferably in the range of 95 to 99.8 mass %, and more preferably in the range of 99 to 99.6 mass %. Such contents are understood in a vaccine composition not containing the solvent.

The content of the MOF in the immunogenic composition may notably depend on the nature of the antigen, and notably its weight and/or its purity.

Therefore, according to another embodiment, the content of the MOF in the immunogenic composition may be, for example, in the range of 70 to 99.9 mass %, preferably in the range of 75 to 99.8 mass %, and more preferably in the range of 85 to 99.6 mass %.

According to another embodiment, the content of the MOF in the immunogenic composition may be, for example, in the range of 3 to 99.9 mass %, preferably in the range of 4 to 99.8 mass %, and more preferably in the range of 5 to 99.6 mass %, for example when bacteria are implemented.

The immunogenic composition according to one embodiment of the present invention may further contain other adjuvant(s) or immune orienters than the MOF.

The immunogenic composition may also contain immunostimulant(s) such as a TLR ligand, an RLR ligand, an NLR ligand, a cyclic dinucleotide or a cytokine.

### Antigen

An immunogenic composition according to the invention contains at least one antigen which is immobilized at least within said Metal-Organic Framework.

According to an embodiment, the immunogenic composition according to the invention may further comprise at least one antigen that is not immobilized within the Metal-Organic Framework.

Suitable antigens that may be used in an immunogenic composition or in a vaccine composition, are described below.

The term “antigen” comprises any molecule, for example a peptide, a protein, a polysaccharide or a glycoconjugate, which comprises at least one epitope that will elicit an immune response and/or against which an immune response is directed. For example, an antigen is a molecule which, optionally after processing, induces an immune response, which is for example specific for the antigen or cells expressing the antigen.

Indeed, according to the present invention, “antigen” means any compound that can and/or that is able to produce an antigen. In particular, an antigen may be chosen from proteins, polysaccharides and their lipidic derivatives, such as polyosides, lipids, molecules obtained by polymerization of amino acids, nucleic acids (natural or modified) coding for an antigen, replicative or non-replicative nucleic acids, coding for antigen, viruses, pseudo-viruses, vaccines, plasmids, phages, etc. or modifying the immune response towards.

According to the present disclosure, any suitable antigen may be envisioned which is a candidate for an immune response. An antigen may correspond to or may be derived from a naturally occurring antigen. Such naturally occurring antigens may include or may be derived from allergens, viruses, bacteria, fungi, parasites and other infectious agents and pathogens or an antigen may also be a tumor antigen. Said antigens may be proteins or peptides antigens, polysaccharide antigens or glycoconjugate antigens.

In other words, the term antigen according to the invention includes antigen, pro-antigen, antigen inducing molecule or an association of more than one antigen, or a molecule able to drive an immune response into a given type. Thus, an antigen according to the invention may act as any direct or indirect specific immune response inducer.

Antigen-containing compositions of the disclosure may vary in their valence. Valence refers to the number of antigenic components in the composition. In some embodiments, the compositions are monovalent. They may also be compositions comprising more than one valence such as divalent, trivalent or multivalent composition.

Antigen-containing compositions of the disclosure may be used as immunogenic compositions and in particular as vaccine compositions, to protect, treat or cure infection arising from contact with an infectious agent, such as bacteria, viruses, fungi, protozoa and parasites.

According to one embodiment, an antigen suitable herein may be selected in the group consisting of bacterial antigens, protozoan antigens, viral antigens, fungal antigens, parasite antigens or tumor antigens.

In another embodiment, wild type or recombinant antigens, or fragments or subunits thereof may be used. Said antigens may be proteins, peptides, polysaccharides and/or glycocongugates.

Preferably, the antigen is chosen from proteins, polyosides, lipids, nucleic acids, viruses, bacteria, parasites, and mixtures thereof, and in particular from tetanus toxoid, a protein derived from SARS-CoV-2 virus, inactivated Escherichia coli, inactivated poliomyelitis virus and meningococcal polysaccharides, and mixtures thereof.

### Bacterial Antigens

The bacterial antigen may be from Gram-positive bacteria or Gram-negative bacteria. Bacterial antigens may be obtained from Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli 0157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Proteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, or Yersinia pestis.

### Viral Antigens

Viral antigens may be obtained from adenovirus; Herpes simplex, type 1; Herpes simplex, type 2; encephalitis virus, papillomavirus, Varicella-zoster virus; Epstein-barr virus; Human cytomegalovirus (CMV); Human herpesvirus, type 8; Human papillomavirus; BK virus; JC virus; Smallpox; polio virus, Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; yellow fever virus; dengue virus; West Nile virus; Rubella virus; Hepatitis E virus; Human immunodeficiency virus (HIV); Influenza virus, type A or B; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus (RSV); Human metapneumovirus; Hendra virus; Nipah virus; Rabies virus; Hepatitis D; Rotavirus; Orbivirus; Coltivirus; Hantavirus, Middle East Respiratory Coronavirus; SARS-Cov-2 virus; Chikungunya virus; Zika virus; parainfluenza virus; Human Enterovirus; Hanta virus; Japanese encephalitis virus; Vesicular exanthemavirus; Eastern equine encephalitis; or Banna virus.

In an embodiment, the antigen is from a strain of Influenza A or Influenza B virus or combinations thereof. The strain of Influenza A or Influenza B may be associated with birds, pigs, horses, dogs, humans or non-human primates.

The nucleic acid may encode a hemagglutinin protein or fragment thereof. The hemagglutinin protein may be H1, H2, H3, H4, H5, H6, H7, H8, H9, H10, H1 11, H12, H13, H14, H15, H16, H17, H18, or a fragment thereof. The hemagglutinin protein may or may not comprise a head domain (HA1). Alternatively, the hemagglutinin protein may or may not comprise a cytoplasmic domain.

In certain embodiments, the hemagglutinin protein is a truncated hemagglutinin protein. The truncated hemagglutinin protein may comprise a portion of the transmembrane domain.

In some embodiments, the virus may be selected from the group consisting of H1N1, H3N2, H7N9, H5N1 and H10N8 virus or a B strain virus.

In another embodiment, the antigen may be from CMV. In particular, antigen may be a combination of a pentamer (gH/gL/pUL128/pUL130/pUL131) and a gB.

In another embodiment, the antigen is from a coronavirus such as SARS-Cov-1 virus, SARS-Cov-2 virus, or MERS-Cov virus.

In another embodiment, the antigen may be from RSV. The antigen may be PreF-ferritin. A prefusion RSV F antigen suitable may be as disclosed in WO 2014/160463 A1 or in WO 2019/195316 A1.

### Fungal Antigens

Fungal antigens may be obtained from Ascomycota (e.g., Fusarium oxysporum, Pneumocystis jiroviecii, Aspergillus spp., Coccidioides immitis/posadasii, Candida albicians), Basidiomycota (e.g., Filobasidiella neoformans, Trichosporon), Microsporidia (e.g., Encephalitozoon cuniculi, Enterocytozoon bieneusi), or Mucoromycotina (e.g., Mucor circinelloides, Rhizopus oryzae, Lichtheimia corymbifera).

### Protozoan Antigens

Protozoan antigens may be obtained from Entamoeba histolytica, Giardia lambila, Trichomonas vaginalis, Trypanosoma brucei, T. cruzi, Leishmania donovani, Balantidium coli, Toxoplasma gondii, Plasmodium spp., or Babesia microti.

### Parasitic Antigens

Parasitic antigens may be obtained from Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa loa, Paragonimus, pinworm, Plasmodium falciparum, Schistosoma, Strongyloides stercoralis, mite, tapeworm, Toxoplasma gondii, Trypanosoma, whipworm, or Wuchereria bancrofti.

### Tumour Antigens

In one embodiment, an antigen may be a tumor antigen, i.e., a constituent of cancer cells such as a protein or peptide expressed in a cancer cell. The term “tumor antigen” relates to proteins that are under normal conditions specifically expressed in a limited number of tissues and/or organs or in specific developmental stages and are expressed or aberrantly expressed in one or more tumor or cancer tissues. Tumor antigens include, for example, differentiation antigens, such as cell type specific differentiation antigens, i.e., proteins that are under normal conditions specifically expressed in a certain cell type at a certain differentiation stage and germ line specific antigens. For example, a tumor antigen is presented by a cancer cell in which it is expressed.

For example, tumor antigens include the carcinoembryonal antigen, a 1-fetoprotein, isoferritin, and fetal sulphoglycoprotein, cc2-H-ferroprotein and γ-fetoprotein.

Other examples for tumor antigens that may be useful in the present invention are p53, ART-4, BAGE, beta-catenin/m, Bcr-abL CAMEL, CAP-1, CASP-8, CDC27/m, CD 4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gapl OO, HAGE, HER-2/neu, HPV-E7, HPV-E6, HAST-2, hTERT (or hTRT), LAGE, LDLR/FUT, MAGE-A, such as MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1 R. Myosin/m, MUC1, MUM-1, -2, -3, NA88-A, NF1, NY-ESO-1, NY-BR-1, pl 90 minor BCR-abL, Pm 1/RARa, PRAME, proteinase 3, PSA, PSM, RAGE, RU1 or RU2, SAGE, SART-1 or SART-3, SCGB3A2, SCP 1, SCP2, SCP3, SSX, SURVIVIN, TEL/AML1, TPI/m, TRP-1, TRP-2, TRP-2/1NT2, TPTE and WT, such as WT-1.

Only one type of antigen may be used, or two or more types thereof may be used in combination.

The content of the antigen in the vaccine composition is, for example, in the range of 0.1 to 10 mass %, preferably in the range of 0.2 to 5 mass %, more preferably in the range of 0.4 to 1 mass %. Such contents are understood in a vaccine composition not containing the solvent.

The content of the antigen in the vaccine composition may notably depend on the nature of the antigen, and notably its weight and/or its purity.

In one embodiment, an immunogenic composition as disclosed herein is a subunit immunogenic composition, for example a subunit vaccine composition.

An immunogenic or vaccine composition as disclosed herein may be formulated into preparations in solid, semi-solid, liquid forms, such as tablets, capsules, powders, aerosols, needles, nanoneedles, suspensions, or emulsions. Typical routes of administering such compositions include, without limitation, oral, topical, transdermal, inhalation, parenteral, sublingual, buccal, intranasal. The term parenteral as used herein includes subcutaneous injections, intravenous, intramuscular, intradermal, intrasternal injection or infusion techniques. In some embodiments, a vaccine composition as disclosed herein may be administered by transdermal, subcutaneous, intradermal or intramuscular route. Compositions of the present disclosure are formulated based upon the mode of delivery, including, for example, compositions formulated for delivery via parenteral delivery, such as intramuscular, intradermal, or subcutaneous injection.

An immunogenic composition as disclosed herein may be administered via any suitable route, such as by mucosal administration (e.g. intranasal or sublingual), parenteral administration (e.g. intramuscular, subcutaneous, transcutaneous, or intradermal route), or oral administration. As appreciated by the man skilled in the art, an immunogenic composition may be suitably formulated to be compatible with the intended route of administration. In one embodiment, an immunogenic composition as disclosed herein may be formulated to be administered via the intramuscular route, or the intradermal route, or the subcutaneous route. In one embodiment, an immunogenic composition may be formulated to be administered via the intramuscular route.

Compositions as disclosed herein are formulated so as to allow the active ingredients contained therein to be bioavailable upon administration of the composition to a subject.

Actual methods of preparing such dosage forms are known, or will be apparent, to those skilled in this art; for example, see Remington: The Science and Practice of Pharmacy, 20th Edition (Philadelphia College of Pharmacy and Science, 2000).

Immunogenic compositions as disclosed herein may be formulated with any pharmaceutically acceptable carrier. The compositions may contain at least one inert diluent or carrier. One exemplary pharmaceutically acceptable vehicle is a physiological saline buffer. Other physiologically acceptable vehicles are known to those skilled in the art and are described, for instance, in Remington's Pharmaceutical Sciences (18th edition), ed. A. Gennaro, 1990, Mack Publishing Company, Easton, Pa. An immunogenic composition as described herein may optionally contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, human serum albumin, essential amino acids, nonessential amino acids, L-arginine hydrochlorate, saccharose, D-trehalose dehydrate, sorbitol, tris (hydroxymethyl) aminomethane and/or urea. In addition, the vaccine composition may optionally comprise pharmaceutically acceptable additives including, for example, diluents, binders, stabilizers, and preservatives.

In one embodiment, the composition may be in the form of a liquid, for example, an emulsion or a suspension. The liquid may be for delivery by injection. Compositions intended to be administered by injection may contain at least one of: a surfactant, preservative, wetting agent, dispersing agent, suspending agent, buffer, stabilizer and isotonic agent may be included. The liquid compositions as disclosed herein may include at least one of: sterile diluents such as water for injection, saline solution, such as physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose.

The pH of an immunogenic composition disclosed herein may range from about 5.5 to about 8, for example from about 6.5 to about 7.5, or may be at about 7.4. Stable pH may be maintained by the use of a buffer. As possible usable buffers, one may cite Tris buffer, HEPES buffer, or histidine buffer. An immunogenic composition as disclosed herein may generally include a buffer. Immunogenic compositions may be isotonic with respect to mammals, such as humans. An immunogenic composition may also comprise one or several additional salts, such as NaCl.

The parenteral preparation can be enclosed in ampoules, disposable syringes or multiple dose vials made of glass or plastic, transdermal high-pressure injectors. An injectable composition is for example sterile.

The compositions as disclosed herein may be prepared by methodology well known in the pharmaceutical art. For example, a composition intended to be administered by injection can be prepared by combining the compositions as disclosed herein with sterile, distilled water or other carrier so as to form a sterile solution or a sterile suspension. A surfactant may be added to facilitate the formation of a homogeneous solution or suspension.

The compositions as disclosed herein are administered in a therapeutically effective amount, which will vary depending on a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the specific disorder or condition; and the subject undergoing therapy.

In one embodiment, immunogenic compositions as disclosed herein may be packaged and stored by any conservation process, for example in dry form such as lyophilized compositions or as micropellets obtained via a prilling process as described in WO 2009/109550.

Dry compositions may include stabilizers such as mannitol, sucrose, or dodecyl maltoside, as well as mixtures thereof, e.g. lactose/sucrose mixtures, sucrose/mannitol mixtures, etc.

### Process for Preparing an Immunogenic Composition

The process for preparing an immunogenic composition according to the invention comprises at least the step consisting to react at least one aluminum compound with at least polycarboxylic acid chosen from fumaric acid, muconic acid, mesaconic acid, oxalic acid, oxaloacetic acid, succinic acid, malic acid, citric acid, aconitic acid, isophthalic acid, substituted isophthalic acid, 2,5-thiophenedicarboxylic acid, 2,5-furandicarboxylic acid, trimesic acid, trimellitic acid or pyromellitic acid and/or with at least polycarboxylate from fumarate, muconate, mesaconate, oxalate, oxaloacetate, succinate, malate, citrate, aconitate, isophthalate, substituted isophthalate, 2,5-thiophenedicarboxylate, 2,5-furandicarboxylate, trimesate, trimellitate and pyromellitate, in the presence of at least one antigen, for forming at least one Al-polycarboxylate Metal-Organic Framework immobilizing said antigen.

Preferably, the aluminum compound is aluminum sulfate.

According to the invention, the aluminum compound can react with said at least polycarboxylic acid and/or with said at least polycarboxylate. The reaction can thus be performed with a polycarboxylate which has been deprotonated separately or with an aluminum precursor. Such step may thus be performed before or during the process according to the present invention.

Preferably, the process according to the invention comprises at least the step consisting to react at least one aluminum compound with at least polycarboxylic acid chosen from fumaric acid, muconic acid, mesaconic acid, oxalic acid, oxaloacetic acid, succinic acid, malic acid, citric acid, aconitic acid, isophthalic acid, substituted isophthalic acid, 2,5-thiophenedicarboxylic acid, 2,5-furandicarboxylic acid, trimesic acid, trimellitic acid or pyromellitic acid, and preferably with at least fumaric acid.

Preferably, the molar ratio of the aluminum compound used for the reaction to polycarboxylic acid and/or polycarboxylate varies from 0.001 to 2.5, preferably from 0.1 to 1.5, preferably from 0.1 to 1, preferably from 0.4 to 0.8, and more preferably from 0.4 to 0.6.

Particular preference is given to a molar ratio of 0.5.

The reaction in the process of the invention is carried out in the presence of an aqueous solvent (aqueous medium).

Here, the water content is, if mixtures are used, preferably more than 50% by weight, more preferably more than 60% by weight, even more preferably more than 70% by weight, even more preferably more than 80% by weight, even more preferably more than 90% by weight, even more preferably more than 95% by weight, even more preferably more than 99% by weight. In particular, the aqueous solvent consists exclusively of water.

In addition, or as an alternative, a base can be used in the reaction.

The reaction is typically carried out in water as solvent in the presence of a base.

Preference is given to using an alkali metal hydroxide or a mixture of a plurality of different alkali metal hydroxides as base. Examples are, in particular, sodium hydroxide and potassium hydroxide. However, further inorganic hydroxides or carbonates or organic bases such as amines are also conceivable. Sodium hydroxide is particularly preferred.

Preferably, the reaction is carried out in the presence of a base, and preferably one alkali metal hydroxide or a mixture of a plurality of different alkali metal hydroxides, and more preferably sodium hydroxide.

In particular, the reaction is carried out at an absolute pressure ranging from 1 to 2 bar, and preferably the reaction is carried out at atmospheric pressure. However, slightly super atmospheric or subatmospheric pressure can occur as a result of the apparatus. For the purposes of the present invention, the term “atmospheric pressure” therefore refers to the pressure range given by the actual prevailing atmospheric pressure 1013 mbar.

According to another embodiment, the reaction may be carried out at 2 bar.

The suitable pressure will be chosen by the man skilled in the art according to the selected antigen, and in any case will preserve the integrity of the medium, and in particular the integrity of the antigen.

The reaction can be carried out at room temperature (20° C.). However, the reaction can take place at temperatures above room temperature.

Preferably, the reaction is carried out at a temperature ranging from 4° C. to 75° C. in particular from 4° C. to 70° C., for example from 4° C. to 65° C., in particular from 10° C. to 70° C., preferably from 10° C. to 45° C., and more preferably from 10° C. to 40° C.

However, the process can be performed at negative temperature, providing the medium composition avoid freezing.

Furthermore, it is advantageous for the reaction to be carried out with mixing of the reaction mixture. The reaction can therefore take place with stirring, which is also advantageous in the case of a scale-up. More effective mixing can be carried out by pumped circulation during the reaction. This makes continuous operation of the process of the invention possible.

To achieve a high space-time yield, the reaction takes place for from 1 min to 96 hours. The reaction is preferably carried out for from 2 hours to 48 hours. The reaction is more preferably carried out for from 5 hours to 24 hours. The reaction is more preferably carried out for from 8 hours to 16 hours.

The molar ratio of polycarboxylic acid and/or polycarboxylate used for the reaction to base used, if the latter is used, is preferably in the range from 0.05 to 2. Greater preference is given to a range from 0.1 to 1.5, even more preferably from 0.2 to 1.

Preferably, the process according to the invention further comprises a centrifugation step at the end of the reaction, and then optionally a redispersion step.

The process according to the invention may also comprises at least one conventional washing step at the end of the reaction.

The present invention will be better understood by referring to the following examples and figures which are provided for illustrative purpose only and should not be interpreted as limiting in any manner the instant invention.

### FIGURES

FIG. 1 illustrates typical characterization techniques of Al-fumarate; (a) PXRD, (b) FT-IR and (c) TGA.

FIG. 2 illustrates the stability of Al-fumarate in HEPES buffer (20 mM, pH 7.4); (a) PXRD over 4 days, (b) Al³⁺ leaching quantified by ICP-OES over two months, (c) fumaric acid leaching quantified by HPLC over two months and (d) weight percentage of Al-fumarate degradation based on HPLC data over two months.

FIG. 3 illustrates the PXRD diagrams of Al-fumarate biocomposites, in which the biomolecule was added either in the ligand/base solution, the metal salt solution or directly to the reaction mixture; (a) BSA, (b) laccase and (c) Cyt c.

FIG. 4 illustrates (a) Immobilization capacity and (b) Protein leaching of Al-fumarate and Alhydrogel® adjuvants after 4 days storage, using BSA and Cyt c as model biomolecules, quantified by the amounts of biomolecule found in the respective supernatants.

FIG. 5 illustrates the characterizations (a,c) PXRD, (b,d) FT-IR of TT@Al-fumarate vaccines of M0 (a,b) and M1 (c,d) concentrations.

FIG. 6 illustrates (a) TT immobilization efficiency for the M0 and M1 formulations, quantified by the amount of TT detected in the supernatants (not adsorbed TT), using the microBCA protein determination assay, (b) illustrates percentage of TT leached form TT@Al-fumarate and TT@Alhydrogel®, after 1 week of the fabrication of the vaccine formulations, quantified by the amount of TT detected in the supernatants (not adsorbed TT), using the microBCA protein determination assay.

FIG. 7 illustrates the index of Ig and IgG anti TT for the two vaccine formulations, TT@Al-fumarate and TT@Alhydrogel®, used in 4 different concentrations (C0-C3) (a) IgG anti TT Ab and (b) whole Ig and IgG anti TT Ab (anti light chain Elisa). Elisa OD are expressed as index, e.g. the value obtained from an immunized mouse, divided par the value observed in serum from the control non immunized naive mice. Mice were bled at D30.

FIG. 8 illustrates Mean body weight and individual mouse body weight evolution for all Sub-Groups (S-TT@Al-fumarate, M-TT@Alhydrogel® and TT) and Control Group.

FIG. 9 illustrates the IgG anti TT response at D14 or D32 observed in ten paired mice immunized using either TT@Alhydrogel® or TT@Al-fumarate. Direct OD observed in ELISA are depicted.

FIG. 10 illustrates comparison of TT@Al-fumarate and TT@Alhydrogel® Ig responses using serial dilutions of 200 to 3200 of sera from Days 7, 14, 32, 60. a) calibration curve in International Units, b) depicts dilution curves c) and d) comparison of curves obtained from sera at D32 and D60 respectively.

FIG. 11 illustrates IgG anti TT responses 32 days after immunization with, from left to right, 9 months-old TT@Al-fumarate, initial TT@ Al-fumarate (same preparation, same immunization used 9 months before) and freshly prepared TT@Al-fumarate.

FIG. 12 illustrates (a) TT immobilization efficiency at the surface of Al-fumarate, quantified by the amount of TT detected in the supernatant (not adsorbed TT), using the microBCA protein determination assay, (b) percentage of TT leached form TT@ Al-fumarate-Surf and TT@Alhydrogel®, after 1 week of the fabrication of the vaccine formulations, quantified by the amount of TT detected in the supernatants (not adsorbed TT), using the microBCA protein determination assay.

FIG. 13 illustrates ζ-potential measurements of TT@Al-fumarate, and TT@ Al-fumarate-Surf formulations, Al-fumarate and that of TT in H₂O.

FIG. 14 illustrates IgG anti TT response 32 days after immunization with, from left to right, TT, TT@Alhydrogel®, TT@Al-fumarate or TT@ Al-fumarate-Surf.

FIG. 15 illustrates the Al³⁺ amounts present at the injection sites of mice (right limbs) at day 7 to day 60 after injection with a log x axis, as quantified by ICP-OES as well as the Al³⁺ wt % present at the injection sites, deducted from the ICP-OES data.

FIG. 16 illustrates the % wt of Al³⁺ present at the injection sites of mice (right limbs) at day 7 to day 60 after injection with a linear x axis, as quantified and deducted from ICP-OES data.

FIG. 17 illustrates the PXRD diagram of fluo-TT@Al-fumarate.

FIG. 18 illustrates the % of initial fluorescence radiance at the injection site over time for mice injected with fluo-TT and fluo-TT@Al-fumarate. Each point represents the mean value of three mice.

FIG. 19 shows HES staining of tissues from organs of naïve mice (top row) and injected mice with TT@Al-fumarate (bottom row). Scale bars represent 500 μm.

FIG. 20 illustrates PXRD diagrams of TT@ZIF-8 and ZIF-8 experimental and calculated.

FIG. 21 illustrates Ig anti-TT obtained 1 month after immunization for TT, TT@Al-fumarate and TT@ZIF-8.

FIG. 22 illustrates the PXRD patterns of formaldehyde inactivated E. coli@Al-fumarate, Al-fumarate experimental and calculated (obtained from CCDC, deposition number: 1051975, database identifier: DOYBEA).

FIG. 23 illustrates TEM images of stained inactivated E. coli (not immobilized, top images) and inactivated E. coli@Al-fumarate (bottom images).

FIG. 24 illustrates STEM-EDX mapping of Al and O on formaldehyde inactivated E. coli@Al-fumarate.

FIG. 25 illustrates the flow cytometry analysis on inactivated E. coli (not immobilized, left), inactivated E. coli@Al-fumarate (middle), released inactivated E. coli from E. coli@Al-fumarate (right); top row: axial and side scatters obtained on BD LSR Fortessa™ device and bottom row: direct video imaging performed using a Thermo Fisher Attune™ Cytpix™ on the bacteria gated on the scatters and/or SYTO 9 fluorophore detecting bacterial DNA.

FIG. 26 illustrates Ig anti E. coli in mice immunized with inactivated E. coli@Al-fumarate, inactivated E. coli or inactivated E. coli@Alhydrogel®. Ranked values are depicted in that same order in each group of 5 mice from the lower to the higher Ig response.

FIG. 27 illustrates the PXRD patterns of inactivated-poliovirus@ Al-fumarate, Al-fumarate experimental and calculated (obtained from CCDC, deposition number: 1051975, database identifier:DOYBEA).

FIG. 28 illustrates a) the amount of proteins detected using the microBCA protein determination assay in IMOVAX POLIO solution, the supernatant of the control reaction of Al-fumarate and the supernatant of inactivated-poliovirus@Al-fumarate, b) the immobilization efficiency for the inactivated-poliovirus@Al-fumarate, deducted from the amount found in the supernatants.

FIG. 29 illustrates the PXRD patterns of glycan@Al-fumarate and calculated (obtained from CCDC, deposition number: 1051975, database identifier:DOYBEA).

FIG. 30 illustrates the ¹³C RMN spectra of Al-fumarate and glycan@Al-fumarate.

FIG. 31 illustrates the PXRD patterns of CpG1018@Al-fumarate Al-fumarate experimental and calculated (obtained from CCDC, deposition number: 1051975, database identifier: DOYBEA).

FIG. 32 illustrates the PXRD patterns of CpG1018+TT@Al-fumarate, Al-fumarate experimental and calculated (obtained from CCDC, deposition number: 1051975, database identifier: DOYBEA).

FIG. 33 illustrates the PXRD patterns of BSA@ Al-muconate and Al-muconate of example 25.

FIG. 34 illustrates the PXRD patterns of MIL-160 of example 26.

FIG. 35 illustrates the PXRD patterns of BSA@Al-trimesate and Al-trimesate of example 27.

FIG. 36 illustrates the PXRD pattern of BSA@Al-pyromellitate of example 28.

### EXAMPLES

### Materials and Methods

All biomolecules and chemicals were purchased form commercial sources and used without further purification unless specified otherwise.

Tetanus toxoid protein 2.8 mg/mL, 1428 Lf/mL, 5712 UI/mL was purchased from Creative Biolabs.

Alhydrogel® adjuvant 2% was purchased from InvivoGen.

Bovine serum albumin, Standard Grade, Zeba™ spin desalting column (7 k MWCO, 2 mL), were purchased from Thermo Fisher Scientific.

Cytochrome C from equine heart, ≥95%, Laccase from Trametes versicolor, ≥0.5 U/mg, Aluminum sulfate, USP testing specifications, Fumaric acid USP/NF specifications, Sodium hydroxide Ph. Eur., BP, NF, E524, 98-100.5% specifications, Aluminum Standard for ICP, 995 mg/L, QuantiPro™ BCA Assay Kit, Phosphate buffered saline tablet, HEPES buffer solution, 1 M in H₂O, Hydrochloric acid, 1 mol/L, Ph. Eur., UPS specifications, Nitric acid, 70%, ≥99.999%, Zinc Standard for ICP, 1000 mg/L, Phosphorus Standard for ICP, 1000 mg/L, 37% formaldehyde, paraformaldehyde, 95-100%, 2-methylimadazole, Ph. Sec. Std., Trimesic acid, 95%, 2,5-furandicarboxylic acid, 1,2,4,5-benzene-tetracarboxylic acid were purchased from Sigma-Aldrich.

Zinc acetate was obtained from Fluka.

Aluminum acetate, basic, 90% was obtained from Acros Organics.

Nitric acid 52.5%, AnalaR NORMAPUR® analytical reagent was purchased from VWR.

Sodium chloride, muconic acid were purchased from Alfa Aesar.

Hydrochloric acid, 37%, for analysis ISO was prurchased from Carlo Erba.

Mouse anti-tetanus toxoid ELISA kits, whole Ig (IgG, IgA, IgM) mouse anti E. coli ELISA kits (ref 500-100 ECP) were purchased from Alpha Diagnostics International.

InVivoTag® 680XL Protein Labelling Kit was purchased from Perkin Elmer.

IMOVAX® was obtained from Sanofi Pasteur.

PNEUMOVAX® vaccine was from MSD.

CpG 1018 was obtained from Proteogenix.

### Mice Studies

For all studies, mice were housed collectively in disposable standard cages in ventilated racks under controlled temperature of 21±3° C., humidity between 30%-70%, with light cycle of 12 hours of light/12 hours of dark. Filtered water and autoclaved standard laboratory food for rodent provided ad libitum. Prior to administration mice were anesthetized under volatile anaesthesia (isoflurane and oxygen as a carrier gas).

For all studies, just before animal administration, the vaccines were kept at room temperature for few minutes in order not to administer a cold solution.

Prior to injection, right before filling the syringe, each vaccine was carefully resuspended by vortexing (3 times, about 5 sec each), unless specified otherwise.

For all studies, injections were done with 26 G disposable needles placed on 50 μL Hamilton syringes.

For all studies, whole blood was sampled and used for serum preparation according to standard protocols.

For intermediate sampling, whole blood was sampled by retro-orbital sinus route using capillary tubes (not coated with anticoagulant). For final sampling, just before euthanasia, whole blood was sampled by intra-cardiac puncture under volatile anaesthesia (isoflurane and oxygen as a carrier gas).

### Instrumentation

Powder X-Ray diffractogram were measured on a Siemens D5000 Diffractometer working in Bragg-Brentano geometry [(θ-2θ) mode] by using CuKα radiation, unless specified otherwise.

Inductively coupled plasma optical emission spectroscopy (ICP-OES) was carried out with an Agilent 720 Series with axially viewed plasma. All samples were filtered prior injection in the instrument unless specified otherwise.

Fourier Transform Infrared Spectroscopy (FT-IR) was performed on a ThermoScientificNicolet 6700 FT-IR.

Thermogravimetric analysis (TGA) was performed on a Mettler Toledo TGA/DSC 1, STAR®System apparatus under O₂ flow.

Optical Density of ELISA kits were measured on a single microtiter plate on a dual wavelength Tecan Spark device.

Origin was used as a statistical software.

### LIST OF ABBREVIATIONS

PXRD: Powder X-ray diffraction
FT-IR: Fourier-transform infrared spectroscopy
TGA: Thermogravimetric analysis
ICP-OES: Inductively coupled plasma optical emission spectroscopy
BSA: Bovine Serum Albumin
Cyt c: Cytochrome c
HEPES: 4-(2-hydroxyethyl)-1-piperazineethanesulfonic acid
PBS: Phosphate buffered Saline
BCA: Bicinchoninic acid
SEM: Scanning electron microscopy
TEM: Transmission electron microscopy
STEM-EDS: Scanning transmission electron microscopy coupled with energy dispersive X-ray spectroscopy
HPLC: High-performance liquid chromatography
RT: Room temperature
PFA 4%: Paraformaldehyde 4% solution
Ab: Antibodies
Ig: Immunoglobulin
IgG: Immunoglobulin G
OD: Optical Density
CCDC: The Cambridge Crystallographic Data Centre
NIR: Near Infra-Red
IQR: Interquartile range
SD: Standard Deviation
BKG: Background
IU: International Units

### Example 1

### Synthesis of Al-Fumarate MOF

For the synthesis of Al-fumarate MOF, 700 mg Al₂(SO₄)₃·xH₂O (x˜18) were dissolved in 10 mL milliQ H₂O.

A separate solution, containing 243 mg fumaric acid and 256 mg NaOH in 10 mL milliQ H₂O was prepared and added to the metal salt solution.

An immediate white precipitation was observed, and the mixture was left under stirring at room temperature for 8 hours, at atmospheric pressure.

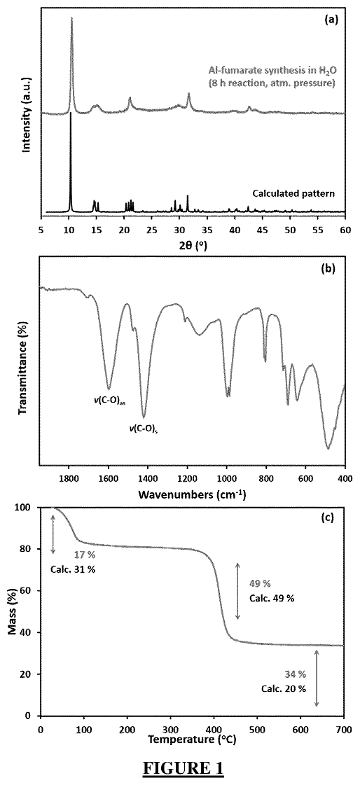

The products were recovered by centrifugation (3 min, 24500 g), dried at 100° C., overnight and analyzed using typical characterization techniques (PXRD, FT-IR, TGA), as illustrated in FIG. 1. The calculated PXRD pattern of Al-fumarate (Basolite® A520) was obtained from The Cambridge Crystallographic Data Centre (CCDC); deposition number: 1051975, database identifier: DOYBEA.

The characterizations were in agreement with the formation of Al-fumarate.

A washing step with milliQ H₂O or HEPES (20 mM, pH 7.4) can be performed if required.

### Example 2

### Stability of Al-Fumarate in HEPES

HEPES buffer at a concentration of 20 mM and pH 7.4 was selected as injection medium for the Al-fumarate adjuvant formulation. The stability of Al-fumarate at the said buffer was studied for a time period of 0 days to 2 months.

More precisely, suspensions with Al-fumarate concentration of 8.5 mg/mL HEPES buffer (20 mM, pH 7.4) were prepared and kept at 4° C., until analysis. The suspensions were manually shaken in various time periods to simulate transportation conditions. At the indicated stability time points, Al-fumarate was collected via centrifugation (20 min, 24 500 g) and dried at 100° C. for 3 hours for analysis. The formulation stability was evaluated on 4 days using typical characterization technique (PXRD) allowing to identify possible structural modifications induced by the buffer. Moreover, ICP-OES and HPLC analytical techniques were employed to evaluate the dissolution of Al-fumarate in the buffer by quantifying the amount of Al³⁺ and fumaric acid leached in solution (supernatant), respectively for up to two months. Note that separate samples were analyzed at each time point.

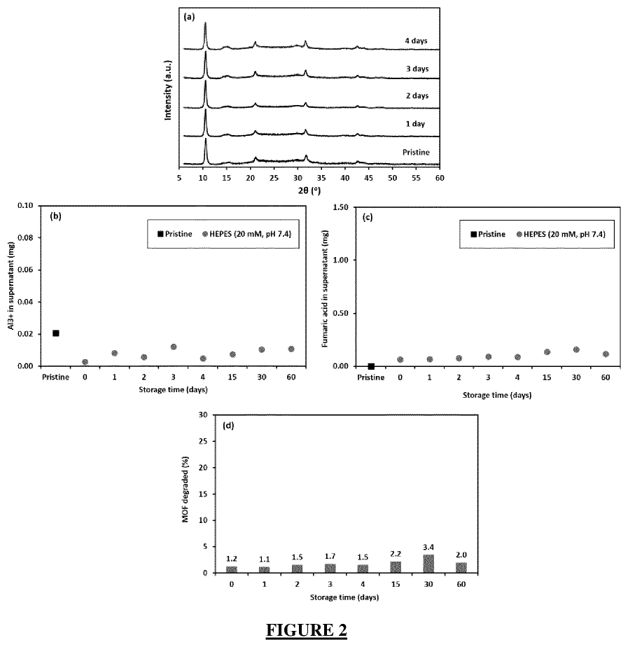

Al-fumarate was found to be stable in HEPES buffer (20 mM, pH 7.4), since no structural modifications were observed over the 4-day time period. Very low quantities of Al³⁺ and fumaric acid were detected in the supernatants over 2 months (FIG. 2), confirming the stability of Al-Fumarate in HEPES buffer (20 mM, pH=7.4) for at least two months.

### Example 3

### Effect of Biomolecule Addition During Al-Fumarate Synthesis

Biomolecule addition during Al-fumarate synthesis was performed using model biomolecules, Bovine Serum Albumin (BSA), Laccase and Cytochrome c (Cyt c), which have different structural characteristics, isoelectric points and sizes.

The way of addition of the biomolecules in Al-fumarate reaction was also examined by either adding the respective biomolecule to the metal salt solution, the ligand/base solution or the reaction, a few seconds after mixing all reactants.

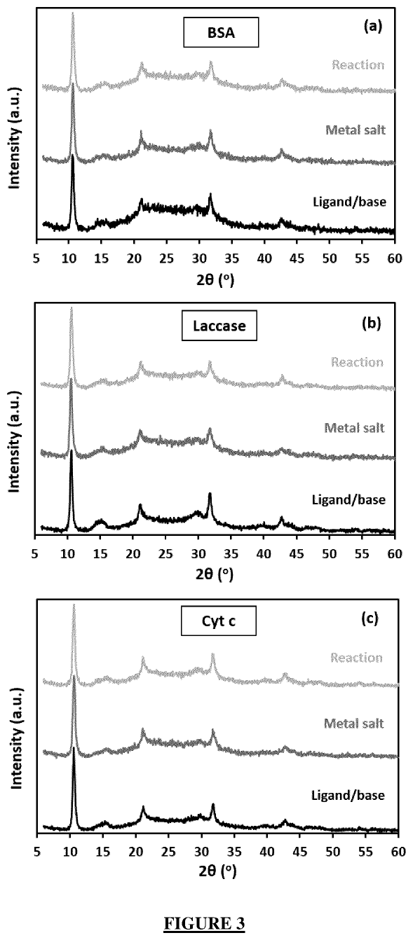

The obtained results showed no influence on the PXRD patterns (FIG. 3) of the biomolecule in the synthesis of Al-fumarate, independently of the way of addition or the structural characteristics of the respective biomolecule.

### Example 4

### Immobilization Capacity of Al-Fumarate and Alhydrogel® Adjuvants

The immobilization capacity of Al-fumarate and Alhydrogel® was investigated using the model biomolecules, Bovine Serum Albumin (BSA) and Cytochrome c (Cyt c).

Additionally, the stability of the biocomposites with both adjuvants was examined for a period of 4 days to determine the quantity of biomolecule leached in solution.

For the Al-fumarate adjuvant, BSA or Cyt c were added to the reaction a few seconds after mixing the metal salt and the ligand/base solutions.

For the Alhydrogel® adjuvant, BSA or Cyt c were mixed with a suspension of the adjuvant for 5 min.

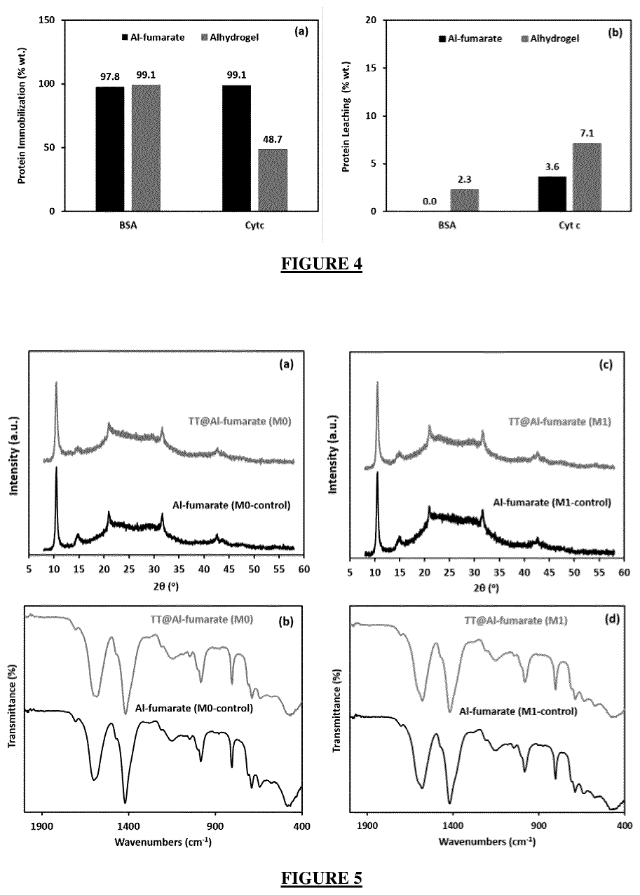

At the end of the respective procedure, the products were centrifuged (3 min, 10 500 g) and the supernatants were collected to quantify the amount of the remaining biomolecule in solution (not adsorbed by the adjuvants), via microBCA protein determination assays (FIG. 4a).

Furthermore, the products were redispersed in HEPES (20 mM, pH 7.4) and PBS (10 mM, pH 7.4) for Al-fumarate and Alhydrogel®, respectively and stored at 4° C., in order to examine the possible leaching of the biomolecules from the adjuvants. At various time periods (between 0 to 4 days), the samples were centrifuged (3 min, 10 500 g) and their supernatants were collected to quantify any biomolecule leached (via microBCA). The samples were again redispersed in the respective buffer and stored at 4° C., until the next measurement up to 4 days. The cumulative amounts up to 4 days of BSA and Cyt c detected in the respective supernatants are shown in FIG. 4b.

Al-fumarate according to the invention demonstrated an excellent immobilization capacity for both tested biomolecules (98% wt. for BSA and 99% wt. for Cyt c), whereas Alhydrogel® was much more efficient for the immobilization of BSA (99% wt.) than Cyt c (49% wt.) (FIG. 4a).

Moreover, the protein leaching studies showed minimal biomolecule quantities were desorbed from Al-fumarate (FIG. 4b).

This study highlights that Al-fumarate is suitable for the immobilization of biomolecules of various characteristics and can have a broad use for the fabrication of different vaccines.

### Example 5

### Immobilization of Tetanus Toxoid in Al-Fumarate

Two TT@Al-fumarate vaccines of different concentrations (M0 and M1) were prepared, while the ratio of TT/Al³⁺ was kept constant to 0.08 IU/Al μg, in agreement with a model human tetanus vaccine.

Tetanus toxoid was also adsorbed on the commercial adjuvant Alhydrogel® at same concentrations (S0 and S1) and ratio (0.08 IU/Al μg) and both vaccine groups were used for in vivo studies.

FIG. 5 shows the PXRD and FT-IR data of the two TT@ Al-fumarate vaccines, compared to those of the control reactions, in which Al-fumarate was formed in absence of antigen, using the exact same reaction conditions as for M0/M1.

As it can be seen, TT did not affect the formation of the MOF, in agreement with the previous studies shown above in example 3, using other biomolecules (BSA, Laccase and Cyt c).

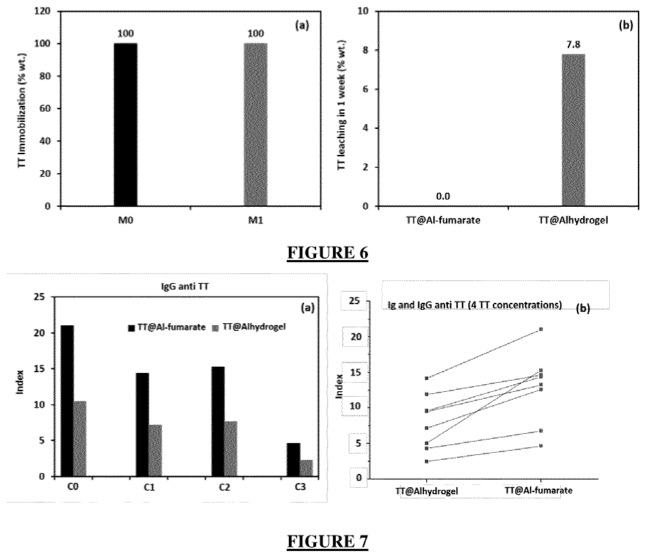

Finally, no Tetanus Toxoid was detected in the supernatants of M0 and M1 using the microBCA protein determination assay, confirming the total immobilization of the antigen in the vaccines (FIG. 6a).

### Example 6

Preparation of Tetanus Toxoid (TT) Antigen Vaccine Compositions with Al-Fumarate or Alhydrogel® and Adjustment of their Doses

The different vaccine compositions and doses were based on a tetanus toxoid monovalent human vaccine and are shown in the table 1 below.

TABLE 1

| | | | Al³⁺ | Antigen | Antigen/Al³⁺ |
|---|---|---|---|---|---|
| | | Dose | content | content | ratio |
| Vaccines | Dose | (μL) | (μg) | (IU) | (IU/μg) |
| Human vaccine | 1 | 500 | 500 | 40 | 0.08 |
| Concentration 0 (C0) | 1/12.5 | 20 | 40 | 3.2 | 0.08 |
| Concentration 1 (C1) | 1/25 | 20 | 20 | 1.6 | 0.08 |
| Concentration 2 (C2) | 1/75 | 20 | 6.7 | 0.5 | 0.08 |
| Concentration 3 (C3) | 1/150 | 20 | 3.3 | 0.3 | 0.08 |

For both adjuvant systems (Al-fumarate and Alhydrogel®), C0 and C1 vaccines were prepared and C1 was used as stock for the C2 and C3 diluted vaccines.

### Immobilization of Tetanus Toxoid (TT) Antigen in Al-Fumarate

For the Al-fumarate-adjuvant vaccines, two TT@ Al-fumarate vaccines M0 and M1 were prepared and M1 was used as stock for the M2 and M3 diluted vaccines. All solutions (reactant, buffer and MilliQ solutions) used were sterilized before use, using Syringe Filters with membranes of 0.2 μm pore size.

The exact quantities used are shown in Table 2 below which shows the experimental details for the preparation of TT@Al-fumarate vaccines.

TABLE 2

| | | | | | | | Tetanus | MilliQ |
|---|---|---|---|---|---|---|---|---|
| TT@Al- | Al³⁺ | Al₂(SO₄)₃•xH₂O | V_{Al-sulfate} | Fumaric | NaOH | V_{fum/NaOH} | toxoid | H₂O |
| fumarate | (mg) | (mg) | (μL) | acid (mg) | (mg) | (μL) | (μL) | (μL) |
| M0 | 0.60 | 7.6 | 109 | 2.6 | 2.8 | 109 | 8.4 | 300 |
| M1 | 0.75 | 9.5 | 136 | 3.3 | 3.5 | 136 | 10.5 | 750 |

For both vaccines, stock solutions of Al₂(SO₄)₃·xH₂O (700 mg) in 10 mL milliQ H₂O and fumaric acid (243 mg)/NaOH (256 mg) in 10 mL milliQ H₂O were used.

The solution of Tetanus Toxoid of 2.8 mg/mL purchased form from Creative Biolabs was used directly for the preparation of the vaccines.

In detail, two separate solutions of 109 μL and 136 μL for M0 and M1, respectively containing either the metal salt or the ligand/base were prepared. A few seconds after mixing the two solutions, the tetanus toxoid solution was added to the reaction (8.4 μL for M0 and 10.5 μL for M1). The final mixture was left under stirring at room temperature for 8 h.

Subsequently, the vaccines were centrifuged at 10 500 g for 3 min, the supernatant was removed and replaced with 300 μL (for M0) or 750 μL (for M1) HEPES buffer (20 mM, pH 7.4).

The TT@ Al-fumarate vaccines were kept at 4° C., for around 2 days until the in vivo studies.

The immobilized quantities of TT in Al-fumarate were quantified based on the amount of TT found in the M0 and M1 supernatants, via microBCA protein determination assay. The amount of TT in M0 and M1 supernatants were negligible, confirming the total immobilization of the TT (FIG. 6a). The Al³⁺ content of the vaccines and their controls was also confirmed by ICP-OES and showed no important variations to the expected values. Table 3 shows the Al³⁺ content of TT@ Al-fumarate vaccines and their controls, quantified by ICP-OES.

Mineralization procedure for ICP-OES: All samples were heated at 100° C. for 16 h, prior to treatment. 1 mL of HCl (1 M) was added to all the dried products, which were then heated in closed vessels at 80° C. for 16 h.

After their complete mineralization, the samples were diluted to 40 mL, with milliQ H₂O for the ICP-OES analysis. A calibration curve of 1000-10,000 ppb Al was used for the analysis.

TABLE 3

| | MOF | Al³⁺ | Al³⁺ | Al³⁺ | Al³⁺ | |
|---|---|---|---|---|---|---|
| | added | theoretical | theoretical | detected by | detected by | MOF by |
| Sample | (mg) | (mg) | (ppb) | ICP (ppb) | ICP (mg) | ICP (mg) |
| M0 | 1.38 | 0.24 | 5876 | 5495 | 0.22 | 1.29 |
| M0-control | 1.10 | 0.19 | 4678 | 4475 | 0.18 | 1.05 |
| M1 | 1.36 | 0.23 | 5801 | 5897 | 0.24 | 1.38 |
| M1-control | 0.81 | 0.14 | 3480 | 3484 | 0.14 | 0.82 |

### Immobilization of Tetanus Toxoid (TT) Antigen in Alhydrogel®

For the Alhydrogel® adjuvant vaccines, two TT@Alhydrogel® vaccines S0 and S1 were prepared and S1 was used as stock for the S2 and S3 diluted vaccines. Buffer solutions used were sterilized before use, using Syringe Filters with membranes of 0.2 μm pore size.

The exact quantities used are shown in Table 4 which details the experimental details for the preparation of TT@Alhydrogel® vaccines.

TABLE 4

| | | Alhy- | | | PBS total |
|---|---|---|---|---|---|
| TT@Alhy- | Al³⁺ | drogel | Tetanus | PBS for TT | (10 mM, |
| drogel ® | (mg) | (μL) | toxoid (μL) | dilution(μL) | pH 7) (μL) |
| S0 | 0.60 | 58.3 | 8.4 | 175 | 300 |
| S1 | 0.75 | 72.8 | 10.5 | 219 | 750 |

The solution of Tetanus Toxoid of 2.8 mg/mL purchased form Creative Biolabs and the adjuvant 2% purchased from InvivoGen were used directly for the preparation of the vaccines.

In detail, 8.4 μL (for S0) or 10.5 μL (for S1) tetanus toxoid solution was diluted in the respective PBS buffer volume (10 mM, pH 7.4), followed by the addition of indicated volume of the Alhydrogel® suspension.

The mixture was pipetted up and down for 5 min, to allow the adsorption of the antigen, and finally the remaining amount of PBS buffer was added.

The TT@Alhydrogel® vaccines were kept at 4° C., for around 2 days until the in vivo studies.

The Al³⁺ content of the commercial Alhydrogel® was also investigated. The Table 5 below shows the Al³⁺ content of Alhydrogel®, quantified by ICP-OES.

TABLE 5

| | Al³⁺ | Al³⁺ | Al³⁺ | Al³⁺ |
|---|---|---|---|---|
| Alhydrogel | theoretical | theoretical | detected by | detected by |
| (μL) | (mg) | (ppb) | ICP (ppb) | ICP (mg) |
| 72.8 | 0.75 | 18750 | 18073 ± 76 | 0.72 |

### Example 7

### Stability of TT@Al-Fumarate and TT@Alhydrogel® Formulations in Term of Antigen Leaching

The stability of the TT@ Al-fumarate formulation in terms of antigen leaching was investigated by determining the amount of TT leached in solution (supernatant) after 1 week of storage at 4° C. No leaching of TT was observed, confirming the stability of the formulation (FIG. 6b).

The TT@Alhydrogel® formulation showed a ˜8% wt. of TT leached in solution (supernatant) after 1 week of storage at 4° C. (FIG. 6b).

### Example 8

### In Vivo Evaluation of the Immune Responses Induced by Vaccines of Example 6

Seven weeks old Balb/c female mice of ˜18 g were immunized by intra-muscular injection in quadriceps muscle of the hind-leg with 20 μL of either TT@ Al-fumarate or TT@ Alhydrogel®.

Four concentrations were tested, with a constant ratio of TT/Al³⁺=0.08 IU/Al μg, for both Al-fumarate and Alhydrogel® adjuvants.

For each concentration, two mice were used per adjuvant group and two additional mice were included in the study as a control group, which did not receive any vaccine injection (naive mice).

No local reaction was observed at the sites of injection. All mice gained weight during the month following immunization.

The immunized mice and the 2 control mice (naive mice) were sacrificed at one month and bled.

Sera were analysed for whole Antibodies (Ab) responses using an anti-mouse light chain Elisa and for IgG Ab responses using an anti-mouse IgG specific Elisa. Elisa was performed according to manufacturer's instruction.

Readings were recorded at two wavelength 450 and 630 nm for correcting the plaque background variations. Sera were tested at 1:10 and 1:100 dilutions for Ig detection as well as at 1:100, 1:1000 for IgG detection. The 1:2500 dilutions were also tested for IgG at the highest Ag concentration. Protein and detergent concentrations were normalized for all serum dilutions used.

Each kit included a reference curve of calibrated samples allowing to express results in kU/mL of Ab.

Index of antibodies (Ab) responses were calculated by dividing the values observed from the immunized mice by the values of sera from the naive mice after subtraction in both of the background of the diluent of the kit (FIG. 7). Whole Ig and IgG Ab responses were evaluated using both Al-fumarate and Alhydrogel® adjuvants. The responses were proportional to the TT and adjuvant concentrations used. At all tested concentration, Al-fumarate induced a statistically significant stronger Ab response that Alhydrogel®.

### Example 9

### Kinetic Study of the Immune Response Triggered by the Two Adjuvanted Vaccines of Example 6 and the Free Antigen

Seven weeks old Balb/c female mice of ˜18 g were immunized by intra-muscular injection in quadriceps muscle of the hind-leg with 20 μL of either TT@Al-fumarate, TT@Alhydrogel® or TT.

The C1 concentration was used (1.6 IU TT/20 μg Al³⁺) for both Al-fumarate and Alhydrogel® adjuvants and 1.6 IU were injected to the mice without an adjuvanted formulation.

For each formulation (TT@Al-fumarate, TT@Alhydrogel® and TT), 24 mice were used per group and two additional mice were included in the study as a control group, which did not receive any vaccine injection (naive mice).

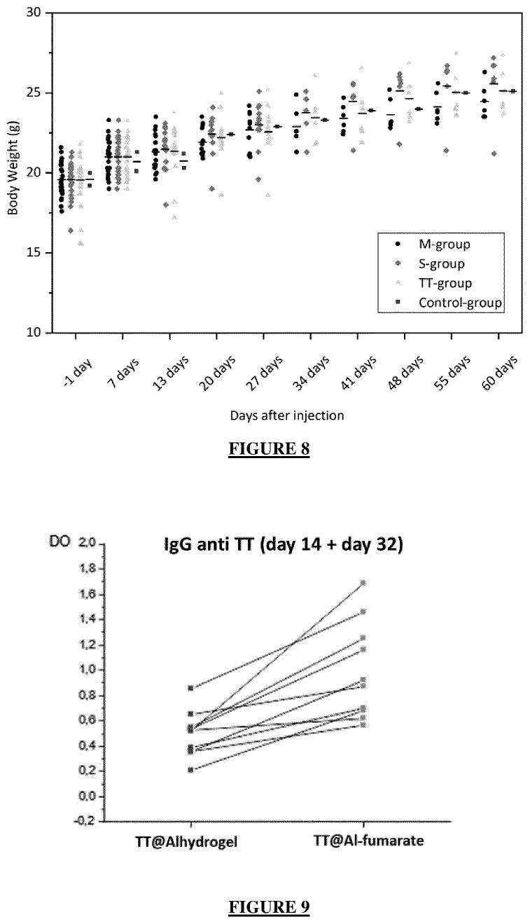

No local reaction was observed at the sites of injection. All mice gained weight during the month following immunization (FIG. 8).

6 mice of each group were sacrificed at 7, 14, 32 and 60 days after injection and sera were collected for ELISA analysis.

The 2 naive mice were sacrificed at day 14 and day 60.

Data from 5 paired mice from day 14 and 5 paired mice from day 32 are depicted in FIG. 9, exhibiting better Ab responses in TT@Al-fumarate injected mice than TT@Alhydrogel® injected mice.

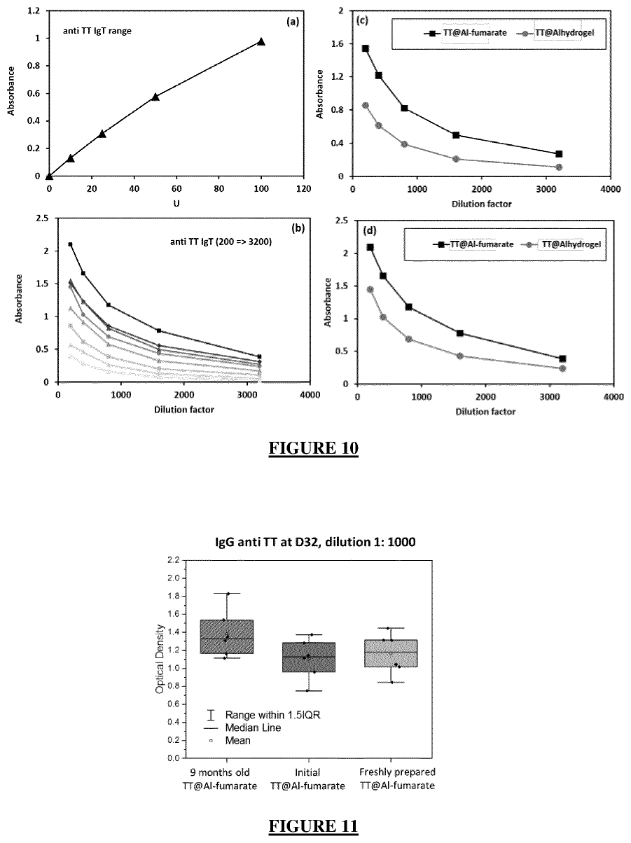

Ab content in serial dilutions 200 to 3200 of sera from Days 7, 14, 32, 60 were analyzed (FIG. 10). At OD of 0.6 (50 U) TT@Al-fumarate or TT@Alhydrogel® Ig led to a dilution ratio TT@Al-fumarate to TT@Alhydrogel® of 3 (20 kU/60 kU) at D32 and 2.5 (40 kU/100 kU) at D60, respectively.

### Example 10

### Long Term Stability of TT@Al-Fumarate in Term of Immunogenic Efficiency

To demonstrate the long term stability of the TT@Al-fumarate vaccine in terms of immunogenicity, a TT@Al-fumarate formulation at the concentration M1 (1.6 IU TT/20 μg Al³⁺) was prepared at the same time than the formulation used in example 9, stored at 4ºC and tested 9 months later. No stabilizer/preservation additives were added to the formulation.

Seven weeks old Balb/cByJ female mice of ˜19 g were immunized by intra-muscular injection in the right hind-limb with 20 μL of either freshly prepared TT@Al-fumarate or 9 months old TT@Al-fumarate (preparation of the solutions are described in example 6).

All vaccines contained 1.6 IU TT/20 μL Al injected. Variation in Al contents between samples were checked by ICP-OES and found less than 8%.

For each formulation (freshly prepared TT@Al-fumarate or 9 months old TT@Al-fumarate), 6 mice were used per group.

The study lasted 32 days, and for all mice, sera were collected 32 days after injection (D32).

No local reaction was observed at the sites of injection. All mice gained weight during the month following immunization, in agreement with the previous study of example 8 and 9.

Sera from this study, as well as the sera obtained 9 months earlier with the same immunogen preparation before aging (example 9), were analysed for whole Antibodies (Ab) responses using an Ig anti-mouse ELISA kit. ELISA was performed according to manufacturer's instruction.

Readings were recorded at two wavelength 450 and 630 nm for correcting the plaque background variations. Sera were tested at 1:1000 dilutions for IgG detection. Protein and detergent concentrations were normalized for all serum dilutions used.

Each kit included a reference curve of calibrated samples allowing expressing results in kU/mL of Ab.

Ab responses were directly expressed as OD. All samples being tested on 3 plates with an identical reference curve on each plate allowing correcting OD values, even though crude reference curve data were nearly identical (1% and 5% variation, respectively).

As shown in FIG. 11, 9 months old TT@Al-fumarate did not show any decrease in its immunogenic properties. The IgG levels were comparable between the aged sample, the freshly prepared one, and those obtained from sera collected 9 months earlier with the same immunogen preparation before aging.

Similar experiments were conducted with TT@Al-fumarate preparation stored at 4° C. without additives for 15 months. The immunogenic properties of the 15 months-old preparation remained in the range of efficacy ≥95% of the freshly prepared TT@ Al-fumarate.

This study highlights that Al-fumarate is suitable for the design of stable vaccine formulation.

### Example 11

Preparation of Tetanus Toxoid (TT) Antigen Vaccine with Surface Immobilization of TT on Al-Fumarate (TT@Al-Fumarate-Surf)

Al-fumarate was tested for the surface adsorption of TT, using the M1 concentration for the formulation. All solutions (reactant, buffer and MilliQ solutions) used were sterilized before use, using Syringe Filters with membranes of 0.2 μm pore size.

The exact quantities used are shown in Table 6 below, which shows the experimental details for the preparation of TT@Al-fumarate-Surf vaccine.

TABLE 6

| Al- | Al³⁺ | Al₂(SO₄)₃•xH₂O | V_{Al-sulfate} | Fumaric | NaOH | V_{fum/NaOH} | H₂O |
|---|---|---|---|---|---|---|---|
| fumarate | (mg) | (mg) | (μL) | acid (mg) | (mg) | (μL) | (μL) |
| M1 | 0.75 | 9.5 | 136 | 3.3 | 3.5 | 136 | 10.5 |

For Al-fumarate synthesis, stock solutions of Al₂(SO₄)₃·xH₂O (700 mg) in 10 mL milliQ H₂O and fumaric acid (243 mg)/NaOH (256 mg) in 10 mL milliQ H₂O were used. In detail, two separate solutions of 136 μL containing either the metal salt or the ligand/base were mixed together. A few seconds after mixing the two solutions, 10.5 μL of H₂O were added to the reaction, corresponding to the volume of TT solution that would have been normally added if preparing a TT@Al-fumarate formulation. The final mixture was left under stirring at room temperature for 8 h.

Subsequently, the product was centrifuged at 10 500 g for 3 min and the supernatant was removed. Al-fumarate was redispersed in 272 μL milliQ H₂O and 10.5 μL of TT solution (2.8 mg/mL purchased form Creative Biolabs) were added for the immobilization procedure.

At the end of the immobilization process (16 h), the TT@Al-fumarate-Surf vaccine was centrifuged at 10 500 g for 3 min, the supernatant was removed and replaced with 750 μL HEPES buffer (20 mM, pH 7.4).

The TT@Al-fumarate-Surf vaccine was kept at 4° C. for further studies.

The immobilized quantity of TT at the surface of Al-fumarate was quantified based on the amount of TT found in the supernatant, via microBCA protein determination assay. As shown in FIG. 12a, the totality of TT was immobilized at the surface of the MOF.

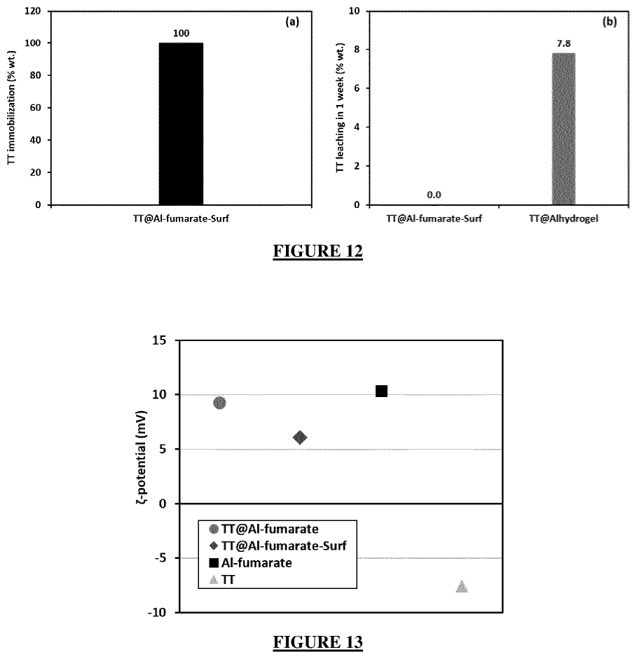

ζ-potential measurements were conducted for the TT@Al-fumarate, TT@Al-fumarate-Surf, Al-fumarate and TT in H₂O, to investigate any changes in the surface charge of Al-fumarate after TT immobilization. As it is shown in FIG. 13, both formulations and the MOF have a positive ζ-potential, whereas TT has a ζ-potential of ˜−8 mV. However, while TT@Al-fumarate and Al-fumarate show similar ζ-potential values (˜9 and 10 mV, respectively), TT@Al-fumarate-Surf has a reduced ζ-potential of ˜6 mV. This difference indicates that for the TT@Al-fumarate formulation, the antigen is entrapped between the MOF particles, whereas for the TT@Al-fumarate-Surf, the antigen is immobilized at the external surface of the MOF, reducing its ζ-potential value, due to the negative charge of TT.

The stability of the TT@Al-fumarate-Surf formulation in terms of antigen leaching was investigated by determining the amount of TT leached in solution (supernatant) after 1 week of storage at 4° C. No leaching of TT was observed from TT@Al-fumarate-Surf, confirming the stability of the formulation, whereas ˜8% wt. of TT was leached form the surface of the Alhydrogel adjuvant (FIG. 12b).

### Example 12

### Evaluation of Vaccines of Example 7 (TT@Al-Fumarate-Surf)

Seven weeks old Balb/cByJ female mice of ˜19 g were immunized by intra-muscular injection in the right hind-limb with 20 μL of either TT, TT@Alhydrogel®, TT@Al-fumarate or TT@ Al-fumarate-Surf.

All vaccines contained 1.6 IU TT/20 μL injected (see example 6 and 11).

The Al³⁺ content of the three aluminum adjuvants were confirmed by ICP-OES. Mineralization procedure for ICP-OES: 200 μL of TT@Alhydrogel®, TT@Al-fumarate or TT@Al-fumarate-Surf vaccines suspensions were heated at 100° C. overnight, prior to treatment. 1 mL of HCl (37%) was added to all the dried products, which were then heated in closed vessels at 80° C. for 16 h. After their complete mineralization, the samples were diluted to 5 mL, with milliQ H₂O for the ICP-OES analysis. Samples were not filtered prior to injection in the instrument.

As shown in table 7, the three vaccines exhibited similar aluminum content.

TABLE 7

| | Volume of vaccine | Al³⁺ | Al³⁺ |
|---|---|---|---|
| | suspensions | detected by | detected by |
| Sample | analyzed (μL) | ICP (ppb) | ICP (mg) |
| TT@ Alhydrogel ® | 200 | 29 362 | 0.15 |
| TT@Al-fumarate | 200 | 29 786 | 0.15 |
| TT@Al-fumarate-surf | 200 | 28 195 | 0.14 |

For each formulation (TT, TT@Alhydrogel®, TT@Al-fumarate or TT@Al-fumarate-Surf), 6 mice were used per group and two additional mice were included in the study as a control group, which did not receive any vaccine injection (naive mice).

The study lasted 32 days, and for all animals, sera were collected 32 days after injection (D32).

No local reaction was observed at the sites of injection. All mice gained weight during the month following immunization, in agreement with the previous studies of example 8, 9 and 10.

Sera were analysed for whole Antibodies (Ab) responses using an anti-mouse light chain Elisa and for IgG Ab responses using an anti-mouse IgG specific Elisa. Elisa was performed according to manufacturer's instruction (Alpha Diagnostics International).

Readings were recorded at two wavelength 450 and 630 nm for correcting the plaque background variations. Sera were tested at 1:1000 for Ig and IgG detection.

Each kit included a reference curve of calibrated samples allowing to express results in kU/mL of Ab.

IgG Ab responses were evaluated using TT, TT@Alhydrogel®, TT@Al-fumarate or TT@Al-fumarate-Surf (FIG. 14). In accordance with the studies of example 8 and 9, IgG levels obtained with TT@Al-fumarate were significantly higher than those obtained with TT@Alhydrogel®, and those obtained with TT without adjuvant. IgG levels obtained with TT@Al-fumarate-Surf were lower than the levels obtained with TT@Al-fumarate, and similar to the level obtained with the reference adjuvant TT@Alhydrogel®.

### Example 13

### Study of the Resorptive Character of Al-Fumarate In Vitro: Studies in Serum and Plasma

This study was conducted by examining the dissolution of the MOF in serum and plasma.

More precisely, 8.76 mg Al-fumarate were dispersed in 1.7 mL of either serum or plasma and incubated at 37° C. under bidimensional continuous stirring (60×60 rpm) for 1 month. At the end of the month, Al-fumarate was recovered via centrifugation (12 000 g, 20 min) and the supernatants (serum or plasma) were collected for the determination of the Al³⁺ content via ICP-OES.

The Al³⁺-ICP-OES analysis of the supernatants showed that 25.1% wt. and 24.0% wt. of the introduced Al-fumarate was degraded in serum and plasma, respectively after 1 month as detailed in the table 8.

TABLE 8

| | | | | Al³⁺ | Al³⁺ | | |
|---|---|---|---|---|---|---|---|
| | MOF | Al³⁺ | Al³⁺ | detected | detected | MOF | MOF |
| | added | theoretical | theoretical | by ICP | by ICP | by ICP | degraded |
| Sample | (mg) | (mg) | (ppb) | (ppb) | (mg) | (mg) | (%) |
| MOF- | 8.76 | 1.5 | 37424 | 13006 | 0.4 | 2.2 | 25.1 |
| Serum | | | | | | | |
| MOF- | 8.76 | 1.5 | 37424 | 12167 | 0.3 | 2.1 | 24.0 |
| Plasma | | | | | | | |

### Example 14

### Evaluation of the Resorptive Character of Al-Fumarate In Vivo

The resorptive character of the TT@ Al-fumarate formulation was evaluated by quantifying the amounts of remaining Al³⁺ at the injection sites of the mice (right limb) and in the blood circulation and compared to that of the non-resorptive TT@Alhydrogel®.

The presence of Al³⁺ (deriving from the two adjuvants) at the injection sites (right limbs) and in the blood circulation of mice was investigated via ICP-OES. The left limbs of all samples, as well as both limbs of all mice injected with only TT and both limbs of the naïve mouse were also analysed by ICP-OES, as negative controls.

Digestion procedure for limb samples: All limb samples were removed from their storage media (PFA 4% in HEPES buffer 20 mM pH 7.4 or EtOH abs. or HEPES buffer 20 mM pH 7.4) and were dehydrated at 100° C. for 5 h before treatment. After dehydration, the limbs were pre-digested with 2.5 mL HNO₃ (70%, analytical grade) for 3 days at RT, followed by a total digestion at 50° C. for 3 h. For the ICP analysis, all digested samples were diluted to a final volume of 20 mL, using milliQ H₂O. A calibration curve of 50-5,000 ppb Al was used for the analysis.

Digestion procedure for blood samples: All blood samples were dehydrated at 100° C. for 5 h before treatment. After dehydration, the blood samples were pre-digested with 300 μL HNO₃ (70%, analytical grade) for 3 days at RT, followed by a total digestion at 50° C. for 3 h. For the ICP-OES analysis, all digested samples were diluted to a final volume of 5 mL, using milliQ H₂O. A calibration curve of 50-5,000 ppb Al was used for the analysis.

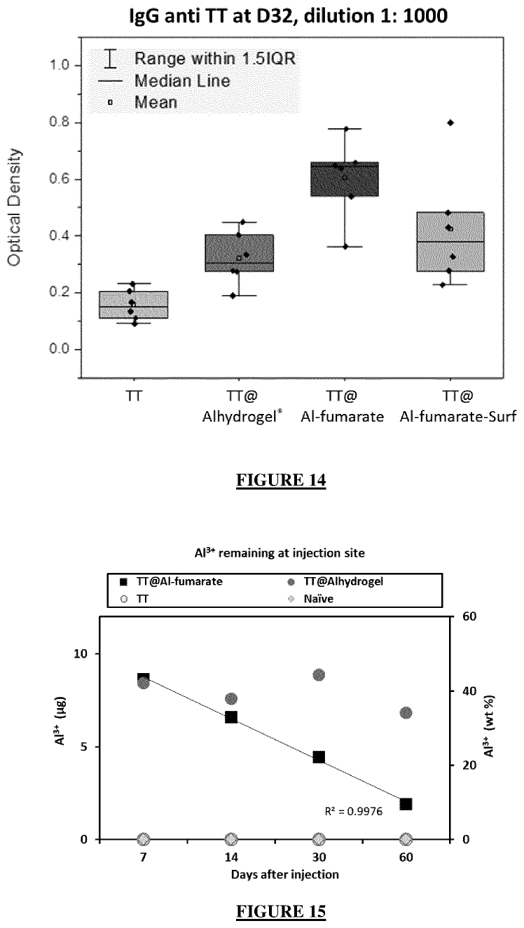

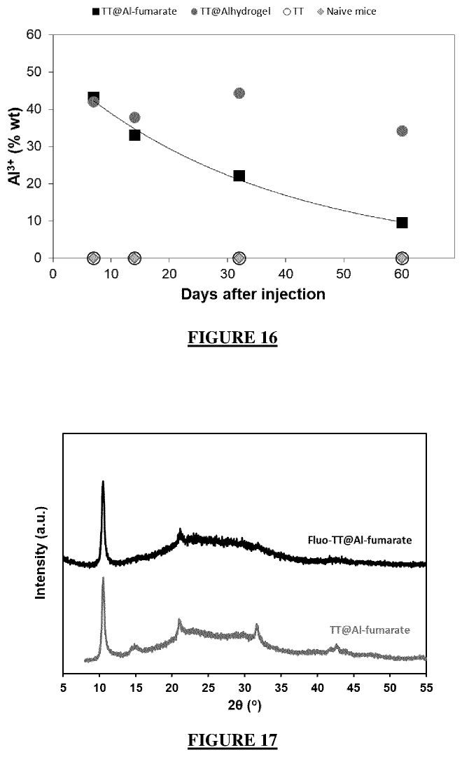

FIGS. 15 and 16 shows the amounts of Al³⁺ quantified by ICP-OES deriving from the digested right limbs of mice from the groups TT@Al-fumarate and TT@Alhydrogel®, as well as the deducted Al³⁺ wt % remaining at the injection site. For both adjuvants, only less than half of the injected Al³⁺ quantity at day 7 (˜9 μg) remained at the injection site. However, starting from day 14, a gradual degradation of the aluminum from TT@Al-fumarate can be observed, whereas the aluminum from TT@Alhydrogel® remains at the injection site as shown by the unchanged amounts of the detected Al³⁺. At day 60, the mice injected with TT@Alhydrogel® presented 3.6 times more Al³⁺ than the mice injected with TT@Al-fumarate. Half-life of aluminum from TT@Al-fumarate was in the range of 25 days whereas aluminum from the TT@Alhydrogel being almost constant displays an apparent half-life of more than 220 days. This study confirms the resorptive character of the TT@Al-fumarate formulation.

No Al³⁺ was detected at the left limbs (not injected limbs) of the TT@Al-fumarate and TT@Alhydrogel® injected mice.

No Al³⁺ was detected either at the right or the left limb of the TT injected mice and at that of the naïve mice.

No Al³⁺ was detected at the blood circulation of any mice group (injected with TT@Al-fumarate or TT@Alhydrogel® or TT, or the naïve mice).

A similar study was conducted with higher injected amount (50 μg Aluminum per hind-limb), and the amount of Al³⁺ detected by ICP-OES at the injection site at day 90 are shown in Table 9. At 90 days, for the mice injected with TT@Al-fumarate only 5% of the injected Al³⁺ remained at the injection site, whereas the mice injected with TT@Alhydrogel® presented 10 times more Al³⁺.

TABLE 9

| | | | % Al³⁺ | |
|---|---|---|---|---|
| | Al³⁺ | Standard | remaining at | Standard |
| | detected by | deviation | the injection | deviation |
| Vaccine | ICP (μg) | (N = 3) | site | (N = 3) |
| TT@Al-fumarate | 2.4 | 1.3 | 4.9 | 2.6 |
| TT@Alhydrogel ® | 26.3 | 6.7 | 52.6 | 13.5 |

### Example 15

### In Vivo Kinetics of Fluorescence Labelled TT@Al-Fumarate

To evaluate the local biodistribution and the persistence at the injection site of TT from TT@Al-fumarate was investigated by time-dependent in vivo NIR imaging using TT labelled with a fluorescent probe, In Vivo Tag 680 NHS fluorescence dye (PerkinElmer).

### Preparation of Fluo-TT@Al-Fumarate

Fluo-TT was prepared by conjugating InVivo Tag 680 XL NHS fluorophore to TT according to manufacturer's instruction. The absence of free remaining dye after Zeba column purification was checked before Fluo-TT encapsulation.

Desalting and fluorochrome conjugation: TT was desalted to NaCl 9:1000 using a 2 mL Zeba Spin column (7 k MWCO). The column was washed twice using 1 mL NaCl 9:1000 by centrifugation at 1 000 g for 3 minutes. 500 μL of TT (2.8 mg/mL) were added in 170 μL, then 130 μL and finally 40 μL NaCl 9:1000 were loaded then centrifuged 3 minutes at 1 000 g. NHS fluorochrome was dissolved in 10 μL DMSO. 4 μL were added to the desalted TT buffered by 50 μL of bicarbonate solution from the labeling kit. After 75 minutes under stirring every 5 minutes, the fluorochrome conjugated TT (fluo-TT) was recovered after column removal of free fluorochrome using the purification column of the kit, previously equilibrated in NaCl 9: 1000 using a 3 minutes 1 000 g centrifugation.

Protein concentration and fluorescence ratio were determined using absorbance measurements at wavelengths of 280 and 668 nm using molar extinction coefficients and equations provided by the kit manufacturer. The resulting fluo-TT solution was at 1 mg/mL (510 Lf/mL, 2 040 UI/mL).

For fluo-TT@Al-fumarate preparation, 81.4 μL of stock solution of Al₂(SO₄)₃·xH₂O (700 mg in 10 mL milliQ H₂O) and 81.4 μL of stock solution of fumaric acid and NaOH (243 mg and 256 mg, respectively in 10 mL milliQ H₂O) were mixed together. A few seconds after mixing the two solutions, 17.64 μL of fluo-TT solution (1 mg/mL) was added to the reaction. The final mixture was left under stirring at room temperature for 8 h. The suspension was centrifuged at 10 000 g for 3 min.

Then, for in vivo studies, the supernatant was removed and replaced with 450 μL HEPES buffer (20 mM, pH 7.4).

For characterization purposes, after centrifugation the obtained powder were dried at 100° C. overnight and the supernatant was collected and analyzed by microBCA assay.

The formation of Al-fumarate in presence of fluo-TT was confirmed by PXRD (FIG. 17). The amount of remaining fluo-TT in the supernatant quantified using the microBCA protein determination assay was found negligible, confirming the almost total immobilization of fluo-TT in Al-fumarate.

This indicates that Al-fumarate is suitable for the immobilization of In Vivo Tag 680-labelled TT.

Fluo-TT vaccine was also prepared, by adding 432 μL HEPES buffer (20 mM, pH 7.4) to 17.64 μL of fluo-TT solution.

Both formulations were kept in the dark at 4° C., for around 6 days until the in vivo studies.

### In Vivo Evaluation of the Presence of Fluo-TT@Al-Fumarate at the Injection Site

Seven weeks old Balb/cByJ female mice of ˜19 g were immunized by intra-muscular injection in the right hind-limb with 50 μL of either fluo-TT or fluo-TT@Al-fumarate.

The formulations were prepared such as all mice were injected with 4 IU (1.96 μg) fluo-TT.

For both fluo-TT and fluo-TT@Al-fumarate formulations, 3 mice were used per group and a naive mouse was also included in the study for background assessment.

Fluorescence acquisitions were performed with the optical imaging system IVIS Spectrum of Perkin Elmer. 2D fluorescence imaging was performed by sensitive detection of light emitted by fluorescent dye (VivoTag680 dye in this study). In vivo fluorescence acquisitions were performed on anesthetized mice with a mixture of isoflurane and oxygen as a carrier gas. During in vivo acquisitions, the animals were placed on the left side (to acquire the fluorescence signal arising from the injection site).

The parameters of in vivo fluorescence imaging are described below:
Field of View (FOV): 14×14 cm (FOV C)
Fluorescent label: In Vivo Tag 680 XL
Excitation wavelength: 640 nm
Emission wavelength: 720 nm
Exposition time: Automatic
Minimum counts: 6000
Binning: between 16 to 4 (automatically adjusted according to the intensity of fluorescence signal)
F/STOP: 2
Subject height: 1.5 cm

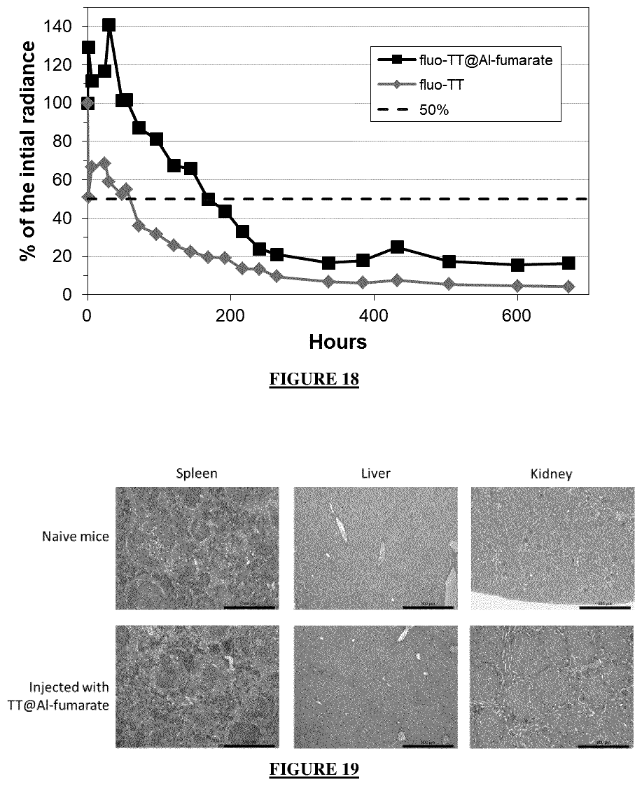

The fluorescence signal was evaluated at different time points after injection as shown on the abscissa of FIG. 18.

Quantification: To calculate the fluorescence signal, a Region of Interest (ROI) was placed on the right mouse hind-limb. The Total radiance efficiency (in p/s/(μ W/cm²) corresponding to the fluorescence signal was obtained for each ROI at each time point.

In case of a second fluorescence acquisition was performed, the Total radiance efficiency was calculated on the second image.

The Total radiance efficiency signal obtained was compared to the mean background reference signal including its standard deviation (BKG+3SD). This reference signal (background radiance efficiency level−BKG radiance efficiency) corresponds to the auto-fluorescence of mice and the noise emitted by the camera of the optical imaging system. It was calculated on the BKG mouse (Group C) according to the following formula:

BKGₗₑᵥₑₗ=meanBKG_{signal(allacquisitions)}+3*BKG_{standarddeviation(allacquisitions)}

All fluorescence signals higher than the BKG radiance efficiency were considered as emitted by injected formulations.

The fluorescence signals (Total radiance efficiency) of each mouse were calculated. The reference autofluorescence signal was measured on the shaved area of the thigh of the control mouse.

A strong quenching of the fluorescent signal within the MOF was observed, the fluo-TT@Al-fumarate signal being half of that of free fluo-TT, in agreement with a full entrapment of the fluo-TT within Al-fumarate.

The radiance signal was normalized to 100% based on the value measured at t=0. More than one Log 10 in radiance signal from fluo-TT@Al-fumarate and control background allowed a longitudinal study for up to 4 weeks.

As shown in FIG. 18, a slower decay of the fluorescence radiance at the injection site was observed in mice injected with fluo-TT@Al-fumarate than using fluo-TT alone.

A 50% fluorescence level was observed after around 60 hours for fluo-TT alone and at an almost three times longer period of time, around 168 hours, for fluo-TT@ Al-fumarate.

Immobilization of the antigen with Al-MOF, and in particular TT in Al-fumarate, leads to a slower release of the antigen at the injection site than without the MOF.

### Example 16

### Evaluation of the In Vivo Toxicity of TT@Al-Fumarate

As indicated in the previous examples (examples 8, 9, 10 and 12), when mice were injected with 20 μL TT@Al-fumarate at concentration C1, i.e. injected with 1.6 IU TT and 20 μg Al³⁺, all mice gained weight during the course of the studies (FIG. 8), indicating the absence of acute toxicity.

To further assess the in vivo toxicity of the TT@ Al-fumarate formulation, higher dose (100 times more) was injected per mice, and the toxicity was evaluated through the evolution of the mice weight, aluminum content in the possible storage organs determined by ICP-OES, and histological analysis of these organs.

Seven weeks old Balb/cByJ female mice of ˜21 g were immunized by intra-muscular injection in both hind-limb with 50 μL and by subcutaneous (SC) in the right flank with 100 μL of TT@ Al-fumarate at concentration M1 (see example 6). The mice were thus in total injected with 200 μg Al and 16 IU TT.

Euthanasia was performed 7 days (1 mouse) and at 32 days (2 mice), 60 days (2 mice) and 90 days (2 mice) after injections.

All mice gained weight during the months following immunization, confirming the absence of acute toxicity.

Non-injected naive mice were used for ICP background checking and normal histological aspect of the tissues and euthanasia were performed at 7, 60 and 90 days.

The organs of interest (spleen, liver) were harvested and either fixed into PFA 4% in HEPES buffer 20 mM pH 7.4 for ICP analysis or in fixative AFA (Alcohol Formalin Acetic Acid) for histological assessment.

The amount of Al³⁺ 60 and 90 days after injection in spleen and liver were analyzed by ICP-OES.

Digestion procedure for the organs: All organs were removed from their storage media (PFA 4% in HEPES buffer 20 mM pH 7.4) and were dehydrated at 100° C. for 5 h before treatment. After dehydration, the organs were pre-digested with 2.5 mL HNO₃ (70%, analytical grade) for 3 days at RT, followed by a total digestion at 50° C. for 3 hours. For the ICP analysis, all digested samples were diluted to a final volume of 20 mL, using milliQ H₂O.

The histological aspect of tissues 7 days after injection in spleen and liver were examined. Kidneys of mice injected with 50 μL Al and naive mice were also examined 35 days after injections.

For histology analysis, tissues were fixed in AFA at least overnight and up to 4 days. The fixed organs were then embedded in paraffin after dehydration in successive baths of ethanol, acetone, and xylene. Each organ was sliced into 5 μM sections, made every 100 μm with a microtome and glued with albuminized glycerine on untreated degreased slide. After paraffin removal, the sections were conventionally stained using HES staining (Hematoxylin, Eosin G and Safranine). The sections were imaged using an optical microscope (Leica DM2000) connected to a digital camera (Leica DF420C), driven by an image acquisition software (LAS V4.2).

As it can be seen in Table 10, the amount of Al³⁺ detected in the spleen and liver at 60 and 90 days after injection was negligible and below 0.6% wt of the injected amount, indicating the absence of accumulation of aluminum in the possible storage organs.

TABLE 10

| | | Al³⁺ detected (μg) | |
|---|---|---|---|
| | Days after | Mice injected with TT@Al- | |
| Organs | injection | fumarate (200 μg Al) | Naive mice |
| Liver | 60 | 0.56 (N = 2) | 0.82 (N = 1) |
| Liver | 90 | 0.04 (N = 2) | |
| Spleen | 60 | 0.24 (N = 1) | 0.22 (N = 1) |
| Spleen | 90 | 0.04 (N = 2) | |

N = number of mice

Images of HES-stained sections from liver and spleen 7 days after injection (200 μg Al), and kidney 35 days after injection (50 μg Al) did not displayed any abnormal aspect on tissue sections (FIG. 19). Particularly, no cellular infiltrates nor abnormal cell morphology were observed. Histological aspects were fully comparable to naïve healthy mice tissues. Kidney tissue sections did not show any glomerular or tubular pathological feature.

This study highlights the absence of acute toxicity, the absence of storage of aluminum in the organism and preserved tissues.

### Example 17

### Evaluation of the Role of Aluminum in the Immunogenicity of TT@Al-Fumarate

To demonstrate the relevance of aluminum, Al-fumarate was compared to a zinc imidazolate MOF, ZIF-8 (ZIF=Zeolitic Imidazolate Framework) in terms of in vivo tetanus toxoid immunogenicity efficiency.

### Preparation of TT@ZIF-8

TT was immobilized within ZIF-8. A stock solution of 2-methylimidazole at 3 mol·L⁻¹ and a stock solution of zinc acetate at 1 mol·L⁻¹ were prepared. 426.5 μL of the 2-methylimidazole stock solution, 10.4 μL milliQ H₂O and 23.1 μL Tetanus Toxoid solution (TT, 2.8 mg/mL, 1 428 Lf/mL, 5 712 UI/mL) were mixed together and vortexed for 10 s. Then, 40 μL of the zinc acetate stock solution was added. The mixture was vortexed for 30 s and then left under stirring at room temperature for 1 hour.

As control experiment, 23.1 μL of milliQ H₂O was added instead of TT solution.

Then, for in vivo studies, the supernatant was removed and replaced with 1 650 μL HEPES buffer (20 mM, pH 7.4).

For characterization purposes, after centrifugation the obtained powders were dry at 100° C. overnight.

The calculated PXRD pattern of ZIF-8 was obtained from the CCDC; deposition number: 602542, database identifier: VELVOY.

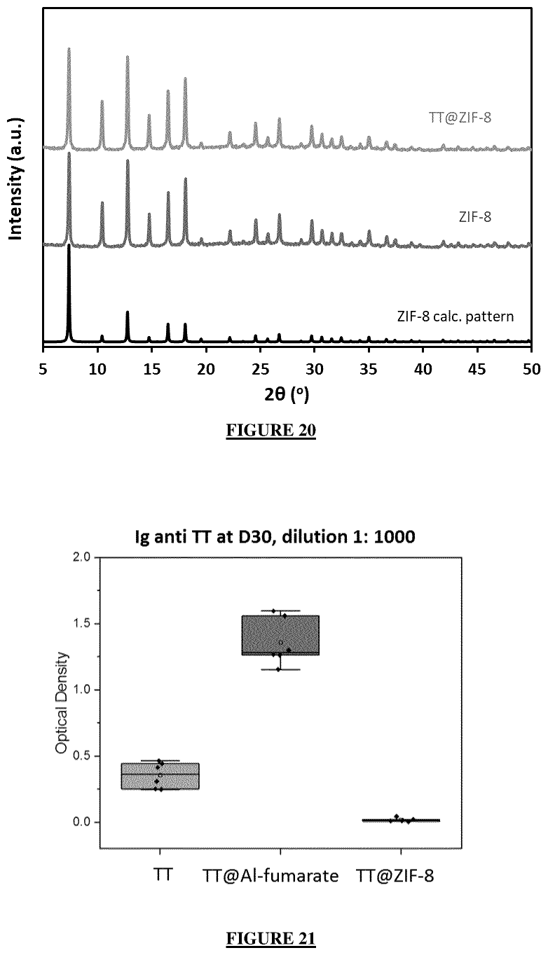

PXRD patterns (FIG. 20) confirmed the formation of ZIF-8 with and without TT.

### In Vivo Evaluation of the Immune Response Triggered by Al or Zn Based Adjuvants.

Seven weeks old Balb/cByJ female mice of ˜19 g were immunized by intra-muscular injection in the right hind-limb with 20 μL of either TT, TT@Al-fumarate or TT@ZIF-8.

TT, TT@Al-fumarate vacines were prepared at the C1 concentration following the same protocols than in example 6.

All vaccines were prepared in the aim to contain 1.6 IU TT/20 μL injected. The metal content of TT@Al-fumarate and TT@ZIF-8 were confirmed by ICP-OES. Mineralization procedure for ICP-OES: 200 μL of each vaccine were heated at 100° C. for 16 h, prior to treatment. 1 mL of HCl (1 M) was added to all the dried products, which were then heated in closed vessels at 80° C. for 16 h. After their complete mineralization, the samples were diluted to 5 mL, with milliQ H₂O for the ICP-OES analysis. Samples were not filtered prior to injection.

As shown in table 11 the two vaccines exhibited similar metal content.

TABLE 11

| | Al | Al³⁺ | Zn | Al³⁺ |
|---|---|---|---|---|
| Sample | detected by | detected by | detected by | detected by |
| (V = 200 μL) | ICP (ppb) | ICP (mg) | ICP (ppb) | ICP (mg) |
| TT@Al- | 30 984 | 0.155 | 7 | 0 |
| fumarate | | | | |
| TT@ZIF-8 | 482 | 0.002 | 36 073 | 0.180 |

For each formulation (TT, TT@Al-fumarate or TT@ZIF-8), 6 mice were used per group and two additional mice were included in the study as a control group, which did not receive any vaccine injection (naive mice).

The study lasted 30 days, and for all animals, sera were collected 30 days after injection (D30).

No local reaction was observed at the sites of injection. All mice gained weight during the month following immunization.

Sera were analysed for whole Ig Antibodies (Ab) responses using an anti-mouse light chain Elisa. Elisa was performed according to manufacturer's instruction (Alpha Diagnostics International). Readings were recorded at two wavelength 450 and 630 nm for correcting the plate background variations. Sera were tested at 1:1000 for Ig detection.

Each kit included a reference curve of calibrated samples allowing to normalize inter plate variations or to express results in kU/mL of Ab.

Ig Ab responses were evaluated using TT, TT@Al-fumarate or TT@ZIF-8 (FIG. 21). The difference in Ig levels between TT and TT@Al-fumarate were in agreement with the previous studies (example 12), with a much higher Ig levels obtained with TT@Al-fumarate. Ig levels obtained with TT@ZIF-8 were negligible, demonstrating the absence of immunization.

This study indicates that ZIF-8 is not suitable for the immobilization of all antigens, and in particular TT.

### Example 18

Immobilization of Formaldehyde Inactivated Escherichia coli in Al-Fumarate

Wild uropathogen E. coli strain with no antibiotic resistance was isolated from a urinary infection on CPSO agar. A few E. coli colonies were recovered and resuspended in 1% aqueous solution of 37% formaldehyde, 1% BSA in PBS buffer (0.150 mM, 7.4). The inactivated E. coli suspension was kept ˜4° C. until use.

Prior to immobilization the inactivated E. coli suspension was washed twice with NaCl 0.9% (2 400 g, 5 min). The resulting suspension was adjusted in order to contain 9-10.10⁶ bacteria/μL (determined by cytometry, see below).

Stock solutions of Al₂(SO₄)₃·xH₂O (700 mg) in 10 mL milliQ H₂O and fumaric acid (243 mg)/NaOH (256 mg) in 10 mL milliQ H₂O were prepared. 1 360 μL of each of the two stock solutions (aluminum precursor and ligand/base) were mixed together. A few seconds after mixing, the inactivated bacteria suspension was added to the reaction (105 μL, 9.6.10⁶ bacteria/μL). The final mixture was left under stirring at room temperature for 8 h. Subsequently, the suspension was centrifuged at 2 000 g for 5 min and washed twice with 0.9% NaCl solution.

The same procedure was also performed without the addition of bacteria, as a control experiment.

The final products were either dried at 100° C. overnight and analyzed using typical characterization techniques (PXRD, TEM) or kept as suspensions at 4° C. for flow cytometry analysis.

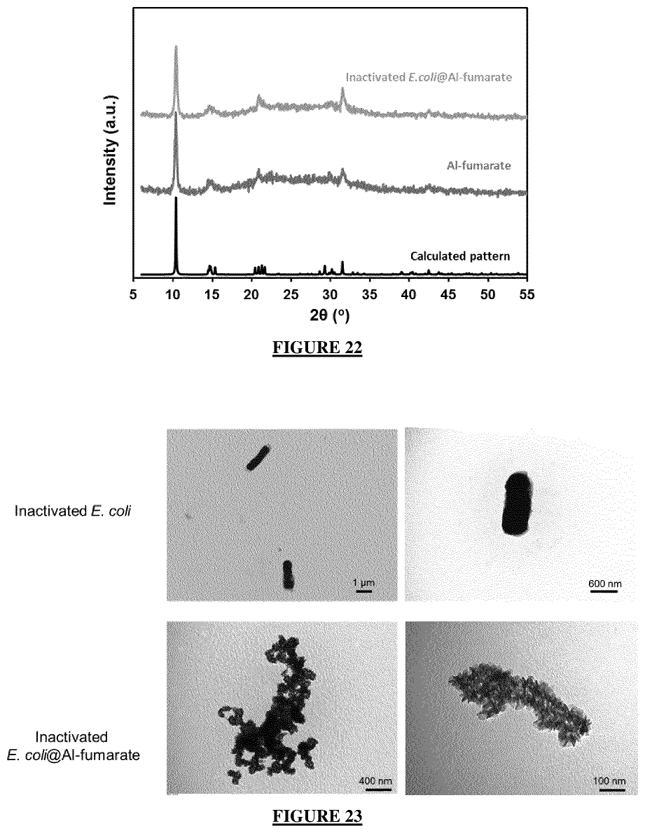

FIG. 22 shows the PXRD patterns of the samples obtained with and without formaldehyde inactivated E. coli, which are both in agreement with the formation of the Al-fumarate MOF.

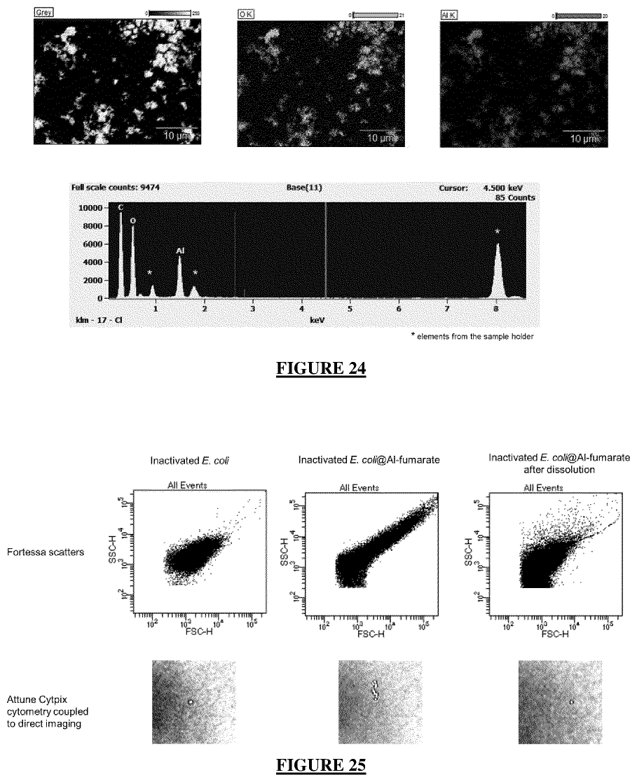

The immobilization of the bacteria was confirmed by TEM images (FIG. 23) and TEM-EDS mapping (FIG. 24).

Imaging procedure: Prior to imaging, the samples were washed twice with H₂O to remove NaCl and avoid its recrystallization on the imaging grid. The samples were diluted to reduce the number of bacteria on the TEM grid. For the TEM grid preparation, one drop of the samples was placed on a carbon-Formar-coated, Cu-mesh TEM grid (EMC). Inactivated bacteria were colorized using a drop of 0.1% phosphotungstic acid (EMC). Once the grids were dried, they were examined using a transmission electron microscope (TEM, Hitachi HT-7700, Japan). Images were taken using a digital camera (Hamamatsu, Japan). STEM-EDS was performed on non-colorized samples on the Hitachi HT-7700 electron microscope equipped with a Bruker x-ray detector.

The TEM images clearly show the presence of particles around the inactivated bacteria (FIG. 23). Unstained STEM-EDS mapping confirmed the presence of Al element homogenously distributed in the sample (FIG. 24).

Formaldehyde inactivated E. coli were analyzed by flow cytometry after bacteria enumeration using True Count™ Becton-Dickinson kit on a BD LSR Fortessa™ and on a Thermo Fisher Attune™ Cytpix™ devices. Axial and side scatters were analyzed on both devices. Direct video imaging was performed using a Thermo Fisher Attune™ Cytpix™ on the bacteria gated on the scatters and/or SYTO 9 fluorophore detecting bacterial DNA.

Bacteria were analyzed in 3 conditions: without MOF, encapsulated in MOF and after dissolution of the MOF after incubating 500 μL of suspension during 3 days in 2 mL 100 mM EDTA, 10 mM PBS pH 7.4.

As shown in FIG. 25, encapsulated bacteria exhibited a different scatter profile than non-encapsulated bacteria, suggesting their coating by the MOF matrix.

On the other hand, bacteria liberated from dissolved MOF exhibited the same scatter profile than non-encapsulated bacteria, indicating their release without morphological damage. This was further confirmed by similar image aspect between non-encapsulated and released bacteria under direct imaging in the Attune™ Cytpix™ Thermo Fisher device.

This study highlights that Al-fumarate is suitable for the immobilization of inactivated bacteria preserving their morphological aspect, and in particular inactivated E. coli.

### Example 19

Preparation of Formaldehyde Inactivated E. coli Antigen Vaccine Compositions with Al-Fumarate or Alhydrogel®

Wild uropathogen E. coli strain with no antibiotic resistance were isolated from a urinary infection on CPSO agar. One E. coli colony was plated on TSA agar. Bulk bacterial culture dish was recovered and resuspended by flooding with 1% aqueous solution of 37% formaldehyde, 1% BSA in PBS buffer (0.150 mM, 7.4). The inactivated E. coli suspension was kept ˜4° C. until use.

Prior to immobilization the inactivated E. coli suspension was washed twice with NaCl 0.9% (2 400 g. 5 min). The resulting suspension was adjusted in order to contain ca 7-8.10⁶ bacteria/μL (determined by flow cytometry).

All solutions (reactant, buffer and MilliQ solutions) used, except bacteria suspension that have been sterilized by formaldehyde fixation, were sterilized before use, using Syringe Filters with membranes of 0.2 μm pore size.

Immobilization of Inactivated E. coli Antigen in Al-Fumarate (E. coli@Al-Fumarate)

To prepare inactivated E. coli@Al-fumarate vaccines, 362 μL of each solution (metal salt and the ligand/base) were mixed together. A few seconds after mixing the two solutions, 28 μL of inactivated E. coli suspension (ca 7.5.10⁶ bacteria/μL) was added to the reaction. The final mixture was left under stirring at room temperature for 8 h. Subsequently, the suspension was centrifuged at 2 400 g for 5 min, the supernatant was removed and replaced with 1 000 μL HEPES buffer (20 mM, pH 7.4).

The inactivated E. coli@Al-fumarate vaccines were kept at 4° C., for around 7 days until the in vivo studies.

Immobilization of Inactivated E. coli Antigen in Alhydrogel

For the Alhydrogel® adjuvant vaccine, the adjuvant 2% purchased from InvivoGen were used directly for the preparation of the vaccines.

28 μL of inactivated E. coli suspension (ca 7.5.10⁶ bacteria/μL) was diluted with 583 μL PBS buffer (10 mM, pH 7.4), followed by the addition of 194 μL Alhydrogel® suspension.

The mixture was pipetted up and down for 5 min, to allow the adsorption of the antigen, and finally 196 μL of PBS buffer was added.

The inactivated E. coli@Alhydrogel® vaccines were kept at 4° C., for around 7 days until the in vivo studies.

Al Content of the E. coli Vaccines

The Al³⁺ content of the inactivated E. coli@Al-fumarate and inactivated E. coli@Alhydrogel® vaccines was investigated by ICP-OES.

Mineralization procedure for ICP-OES: 200 μL inactivated E. coli@Al-fumarate and inactivated E. coli@Alhydrogel® vaccines were heated at 100° C. for 16 h, prior to treatment. 1 mL of HCl (37%) was added to all the dried products, which were then heated in closed vessels at 80° C. for 16 h. After their complete mineralization, the samples were diluted to 5 mL, with milliQ H₂O for the ICP-OES analysis. The samples were not filtered prior to injection in the instrument, and were run in duplicates.

Table 12 shows the Al³⁺ content of the inactivated E. coli vaccines quantified by ICP-OES. As it can be seen, both inactivated E. coli@Al-fumarate and inactivated E. coli@Alhydrogel® vaccines had relatively similar aluminum content.

TABLE 12

| Sample | Al³⁺ detected by | Al³⁺ detected by |
|---|---|---|
| (V analyzed = 200 μL) | ICP (ppb) | ICP (mg) |
| Inactivated E. coli@Al-fumarate | 60 233 | 0.301 |
| Inactivated E. coli@Alhydrogel ® | 62 949 | 0.315 |

Inactivated E. coli Antigen without Adjuvant

For inactivated E. coli vaccine, 28 μL of inactivated E. coli suspension (ca 7.5.10⁶ bacteria/μL) was diluted with 972 μL PBS buffer (10 mM, pH 7.4). The inactivated E. coli vaccines were kept at 4° C., for around 7 days until the in vivo studies.

### Example 20

### Evaluation of the In-Vivo Immune Response of Vaccines of Example 19

Seven weeks old Balb/cByJ female mice of ˜20 g were immunized by intra-muscular injection in the right hind-limb with 50 μL of either inactivated E. coli, inactivated E. coli@Al-fumarate or inactivated E. coli@Alhydrogel®.

As described in example 19, each vaccine was prepared to contain comparable amount of bacteria, with a constant ratio of Al, for both Al-fumarate and Alhydrogel® adjuvants.

For each formulation (inactivated E. coli, inactivated E. coli@Al-fumarate or inactivated E. coli@Alhydrogel®) 10 mice were used per group and 3 additional mice were included in the study as a control group, which did not receive any vaccine injection (naive mice).

Euthanasia was performed for half of the mice (n=5) 21 days after the injection (D21) and for the remaining mice (n=5) 42 days after injection (D42). 1 naïve mouse was sacrificed at 21 days after injection, and 2 naïve mice were sacrificed at 42 days.

21 days after the injection, the remaining mice of each group, received another 50 μL intra-muscular injection in quadriceps muscle of the right hind-leg.

For one animal per group, at the start of the study (DO), blood was sampled by retro-orbital sinus route using dry capillary tubes allowed to clot, then used for serum preparation. For all animals, before euthanasia (D21 or D42), whole blood was sampled by intra-cardiac puncture and used for serum preparation.

All mice gained weight during the 21 or 42 days following immunization.

Whole Ig (IgG IgA IgM) in sera were detected using mouse anti E coli Elisa kits from Alpha Diagnostic International ref 500-100 ECP. Plates are coated with purified lysates of TOP10, K12, DH5a, BL21, HB101 E. coli strains. Sera were tested diluted 1:1000 and according to manufacturer's instruction.

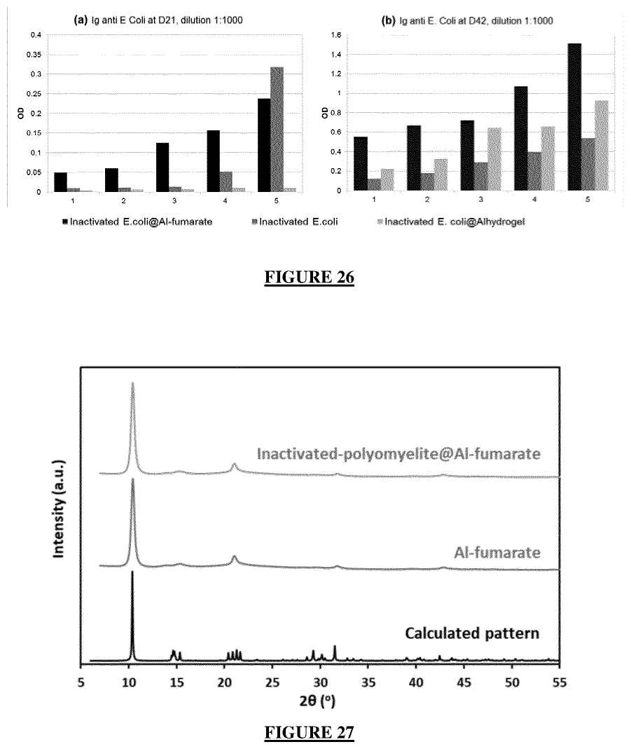

Among sera from D21 (FIG. 26a), all 5 mice injected with inactivated E. coli@Al-fumarate exhibited a strong Ig response whereas Ig level among inactivated E. coli@Alhydrogel® injected mice or inactivated E. coli without adjuvant remaining low, with inactivated E. coli@Al-fumarate ratio index of 16.9 et 1.6, respectively. The smaller ratio was due to an atypical mouse among the no-adjuvant group which exhibited the higher Ig response and a pre-immune level 2.7 fold higher than other non-immunized mice, demonstrating a previous sensitization. The level of Ig of the other no adjuvant mice remained in the very low level of the E. coli@ Alhydrogel® injected mice.

At D42 (FIG. 26b), mice from the inactivated E. coli@Al-fumarate exhibited a higher Ig level than mice from the other groups, 3 time higher than without adjuvant and 1.63 higher than using reference Alhydrogel® adjuvant.

This study highlights that Al-fumarate is suitable for the immobilization of inactivated bacteria preserving their immunogenic potential and acts as adjuvant leading to an enhanced immune response compared to bare inactivated bacteria and even to the reference Alhydrogel® adjuvant.

### Example 21

### Immobilization of Inactivated Polioviruses in Al-Fumarate (Inactivated Polio@Al-Fumarate)

IMOVAX® POLIO vaccine from Sanofi Pasteur was used as a source of inactivated poliomyelitis virus. One dose (0.5 mL) contains inactivated Poliomyelitis virus: Type 1 (Mahoney strain produced on VERO cells) 40 D-antigen Unit (DU), Type 2 (MEF-1 strain produced on VERO cells) 8 DU, Type 3 (Saukett strain produced on VERO cells) 32 DU.

Stock solutions of Al₂(SO₄)₃·xH₂O (700 mg) in 10 mL milliQ H₂O and fumaric acid (243 mg)/NaOH (256 mg) in 10 mL milliQ H₂O were prepared. 1 554 μL of each of the two stock solutions (aluminum precursor and ligand/base) were mixed together. A few seconds after mixing, the IMOVAX® POLIO solution was added to the reaction (120 μL). The final mixture was left under stirring at room temperature for 8 h. The product (inactivated-polyomyelite@Al-fumarate) was recovered by centrifugation (10 000 g, 3 min).

The same procedure was also performed with the addition 120 μL H₂O instead of IMOVAX® POLIO solution, as a control experiment (Al-fumarate).

The final products were dried at 100° C. overnight and analyzed using PXRD characterization technique (FIG. 27).

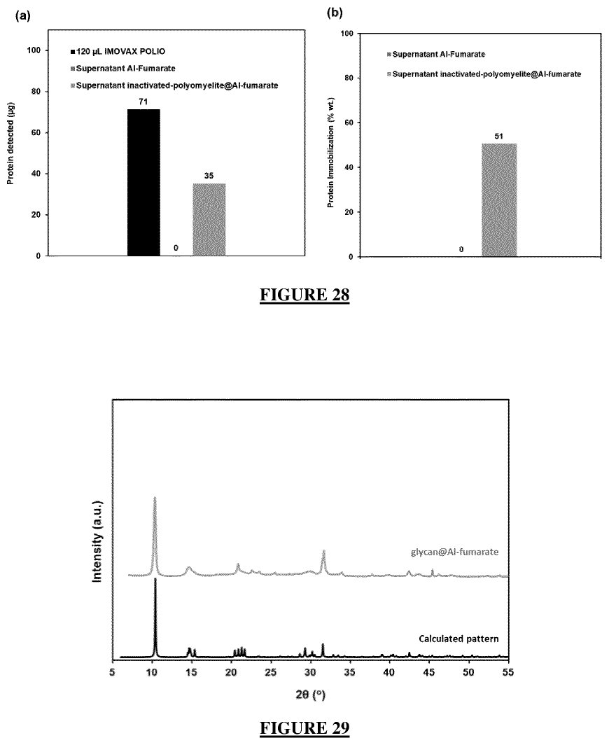

The supernatants were collected to quantify the amount of the remaining proteins in solution (not adsorbed), via microBCA protein determination assays (FIG. 28). As a control for the microBCA protein determination assays, 120 μL IMOVAX® POLIO solution was used.

The obtained PXRD pattern is in agreement with the formation of Al-fumarate in the presence of inactivated poliovirus (FIG. 27).

Al-fumarate according to the invention demonstrated an immobilization capacity of >50% of the introduced inactivated poliovirus suspension based on quantification by microBCA assay of the remaining proteins in the synthesis supernatant (FIG. 28).

This study highlights that Al-fumarate is suitable for the immobilization of inactivated viruses, and in particular inactivated poliomyelitis virus from IMOVAX® POLIO vaccine.

### Example 22

### Immobilization of Glycans in Al-Fumarate (Glycan@Al-Fumarate)

PNEUMOVAX® vaccine from MSD was used as a source of pneumococcal capsular polyoside. One dose (0.5 mL) contains 25 μg of each 23 pneumococcal polysaccharide serotypes (1, 2, 3, 4, 5, 6B, 7F, 8, 9N, 9V, 10A, 11A, 12F, 14, 15B, 17F, 18C, 19F. 19A, 20, 22F, 23F, 33F).

Prior to use, the vaccine solution was lyophilized. Samples were dipped into liquid nitrogen for a few minutes and then lyophilized for 24 h. The resulting powder was dissolved in 50 μL MilliQ H₂O.

Stock solutions of Al₂(SO₄)₃·xH₂O (700 mg) in 10 mL milliQ H₂O and fumaric acid (243 mg)/NaOH (256 mg) in 10 mL milliQ H₂O were prepared. 653 μL of the two stock solutions (aluminum precursor and ligand/base) were mixed together. A few seconds after mixing, 50 μL of the glycan solution (25 μg each/50 μL) was added to the reaction. The final mixture was left under stirring at room temperature for 8 h. The product (glycan@Al-fumarate) was recovered by centrifugation (10 000 g, 3 min).

The same procedure was also performed with the addition 50 μL H₂O instead of glycan solution, as a control experiment (Al-fumarate).

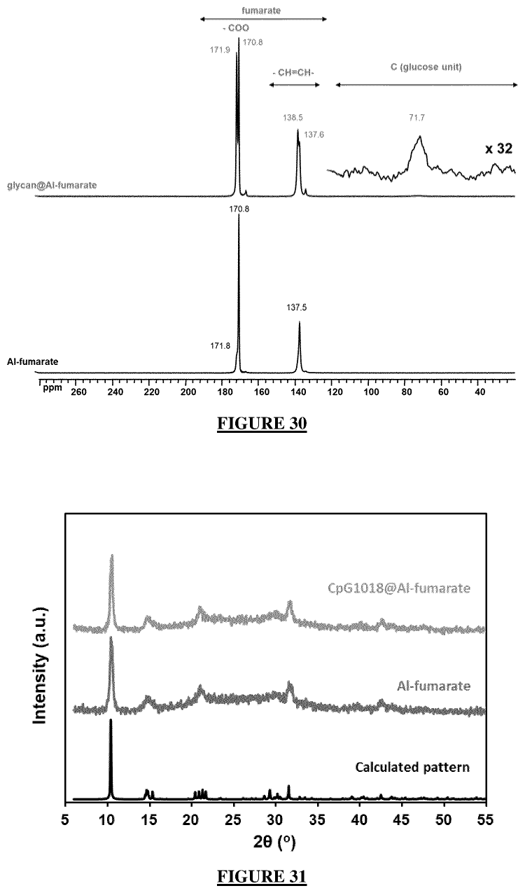

The final products were dried at 100° C. overnight and analyzed using PXRD (FIG. 29) and ¹³C NMR spectroscopy (FIG. 30). PXRD were measured on a Bruker D8 Advance diffractometer with a Debye-Scherrer geometry, equipped with a Ge(111) monochromator selecting Cu Kα1 radiation (λ=1.540598 Å) and a LynxEye detector. Powders were loaded in glass capillaries. ¹³C RMN spectra were recorded on an Advance Bruker 500 MHz NMR spectrometer operating at static magnetic field of 11.7 T, corresponding to Larmor frequencies of 126 MHz for ¹³C. The ¹³C{1H} CPMAS spectra were acquired with 5*0.5 ms contact time, 20 KHz.

The PXRD pattern showed that Al-fumarate was formed in the presence of glycans (FIG. 29).

¹³C NMR spectra (FIG. 30) indicated the presence of Csp3 characteristics of sugar units in the glycan@Al-fumarate sample (delta ˜ 71 ppm), those carbons were not present in Al-fumarate sample. These results confirm the presence of glycan in the MOF powder, indicating their immobilization.

This study highlights that Al-fumarate is suitable for the immobilization of glycan, and in particular those from PNEUMOVAX® vaccine.

### Example 23

### Immobilization of Nucleic Acid (CpG 1018) in Al-Fumarate (CpG1018@Al-Fumarate)

CpG 1018 (phosphorothioate oligonucleotides, 22-mer, sequence: in the powder form and directly used without further purification. TGACTGTGAACGTTCGAGATGA, modification: all bases) was obtained from Proteogenix.

1051.83 μg CpG 1018 was dissolved in 35 μL of MilliQ H₂O.

Stock solutions of Al₂(SO₄)₃·xH₂O (700 mg) in 10 mL milliQ H₂O and fumaric acid (243 mg)/NaOH (256 mg) in 10 mL milliQ H₂O were prepared. 136 μL of the two stock solutions (aluminum precursor and ligand/base) were mixed together. A few seconds after mixing, the CpG solution was added to the reaction (10 μL, 30 μg/μL). The final mixture was left under stirring at room temperature for 8 h. The product was recovered by centrifugation (10 000 g, 5 min).

As control experiment, the same procedure was also performed with the addition of 10 μL H₂O instead of CpG solution.

The final products were dried at 50° C. for 8h and analyzed using PXRD technique and the supernatants were collected to quantify the amount of remaining CpG 1018 in solution (not immobilized).

The immobilization of CpG was investigated by ICP-OES, as CpG 1018 contains P elements whereas Al-fumarate does not contain any P elements.

Mineralization procedure for ICP-OES: All samples were heated at 50° C. for 8 h, prior to treatment. 300 μL of HCl (37%) was added to all the dried products, which were then heated in closed vessels at 80° C. for 16 h. After their complete mineralization, the samples were diluted to 5 mL, with milliQ H₂O for the ICP-OES analysis. The samples were not filtered prior to injection.

As it can been in FIG. 31, the PXRD patterns showed that Al-fumarate was formed in the presence of CpG 1018.

Table 13 below shows the P content of Al-fumarate and CpG1018@Al-fumarate, samples, as well as their respective supernatants detected by ICP-OES.

TABLE 13

| | Amount of | | | |
|---|---|---|---|---|
| | sample | | P detected | P detected |
| Sample | analyzed | | by ICP (ppb) | by ICP (mg) |
| Al-fumarate | 4.48 | mg | 14 | 0 |
| CpG1018@Al-fumarate | 5.88 | mg | 3952 | 0.020 |
| Al-fumarate supernatant | 200 | μL | 12 | 0 |
| CpG1018@Al-fumarate | 200 | μL | 54 | 0 |

supernatant

The absence of P element in Al-fumarate was confirmed as the amount of P detected was negligible in Al-fumarate sample and it supernatant.

P element was found to be negligible in the supernatant of CpG1018@Al-fumarate, suggesting the absence of CpG 1018, whereas P element was detected in CpG1018@Al-fumarate sample, indicating its immobilization with Al-fumarate.

This study highlights that Al-fumarate is suitable for the immobilization of nucleic acid, and in particular CpG 1018.

### Example 24

### Immobilization of Both Nucleic Acid (CpG 1018) and Tetanus Toxoid in Al-Fumarate (CpG1018@Al-Fumarate)

CpG 1018 (phosphorothioate oligonucleotides, 22-mer, Sequence: TGACTGTGAACGTTCGAGATGA, modification: all bases) was obtained from Proteogenix in the powder form and directly used without further purification.

1051.83 μg CpG 1018 was dissolved in 35 μL of MilliQ H₂O.

The solution of Tetanus Toxoid (TT) at 2.8 mg/mL purchased from Creative BIolabs was used directly.

Stock solutions of Al₂(SO₄)₃·xH₂O (700 mg) in 10 mL milliQ H₂O and fumaric acid (243 mg)/NaOH (256 mg) in 10 mL milliQ H₂O were prepared. 136 μL of the two stock solutions (aluminum precursor and ligand/base) were mixed together. A few seconds after mixing, 10 μL of the CpG 1018 solution (30 μg/μL) and 10 μL of the TT solution (2.8 mg/mL) were added to the reaction. The final mixture was left under stirring at room temperature for 8 h. The product (CpG1018+TT@Al-fumarate) was recovered by centrifugation (10 000 g, 5 min).

As control experiments, the same procedure was also performed with the addition of 20 μL H₂O instead of CpG and TT solutions (Al-fumarate), and with the addition of only 10 μL TT solution (TT@Al-fumarate).

The final products were dried at 50° C. for 8h and analyzed using PXRD and the supernatants were collected to quantify the amount of remaining CpG 1018 and TT in solution (not immobilized).

The amount of immobilized TT was investigated by quantifying the amount of the remaining TT in the supernatant (not adsorbed), via microBCA protein determination assays.

The immobilization of CpG was investigated by ICP-OES, as CpG contains P elements.

Mineralization procedure for ICP-OES: All samples were heated at 50° C. for 8 h, prior to treatment. 300 μL of HCl (37%) was added to all the dried products, which were then heated in closed vessels at 80° C. for 16 h. After their complete mineralization, the samples were diluted to 5 mL, with milliQ H₂O for the ICP-OES analysis. The samples were not filtered prior to injection.

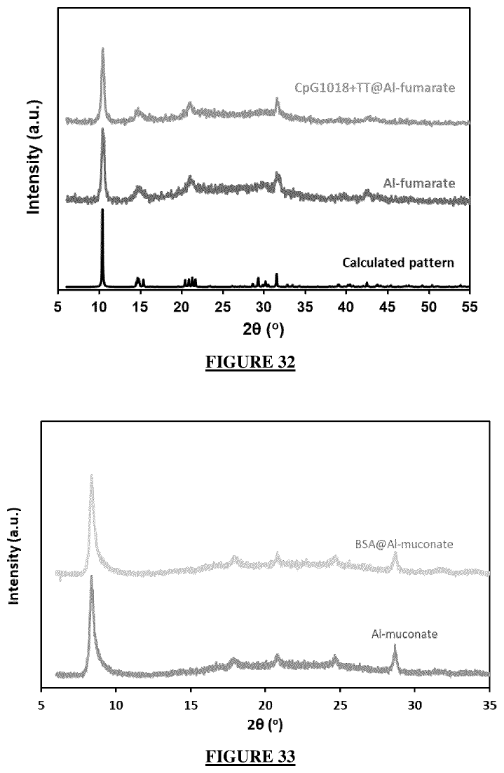

As it can been in FIG. 32, the PXRD patterns showed that Al-fumarate was formed in the simultaneous presence of CpG 1018 and TT.

The amount of immobilized TT in CpG1018+TT@Al-fumarate was found to be >78% of introduced TT, by quantifying the amount of the remaining TT in the supernatant (not adsorbed), via microBCA protein determination assays.

Table 14 below shows the P content of Al-fumarate, TT@Al-fumarate and CpG1018+TT@Al-fumarate samples, as well as their respective supernatants.

P elements were only detected in CpG1018+TT@Al-fumarate and TT@Al-fumarate samples. The amount of P elements detected in TT@Al-fumarate samples was negligible compared to the amount of P elements detected in CpG1018+TT@Al-fumarate samples, indicating that the P elements detected in CpG1018+TT@Al-fumarate sample mainly results from the presence of CpG1018.

These results indicate that CpG 1018 was immobilized with Al-fumarate in presence of TT.

TABLE 14

| | Amount of | | | |
|---|---|---|---|---|
| | sample | | P detected | P detected |
| Sample | analyzed | | by ICP (ppb) | by ICP (mg) |
| Al-fumarate | 5.33 | mg | 9 | 0 |
| Al-fumarate supernatant | 200 | μL | 8 | 0 |
| TT@Al-fumarate | 5.77 | mg | 349 | 0.002 |
| TT@Al-fumarate | 200 | μL | 12 | 0 |
| supernatant | | | | |
| CpG1018 + TT@Al- | 5.05 | mg | 3440 | 0.017 |
| fumarate | | | | |
| CpG1018 + TT@Al- | 50 | μL | 15 | 0 |

fumarate supernatant

This study highlights that Al-fumarate is suitable for the combined immobilization of nucleic acid and proteins, and in particular CpG1018 and Tetanus Toxoid.

### Example 25

Biomolecule Immobilization within Al-Muconate

For the synthesis of BSA@Al-muconate, 700 mg Al₂(SO₄)₃·xH₂O (x˜18) were dissolved in 10 mL milliQ H₂O. A separate solution, containing 297 mg muconic acid (trans, trans-1,3-Butadiene-1,4-dicarboxylic acid) and 256 mg NaOH in 10 mL milliQ H₂O was prepared and added to the metal salt solution. 200 μL BSA (0.15 mg/μL) was immediately added to the mixture and the mixture was left under stirring at room temperature for 20 hours, at atmospheric pressure.

A control experiment was performed with the addition of 200 μL milliQ H₂O instead of BSA solution.

The products were recovered by centrifugation (20 min, 21 200 g), washed 3 times with water and dried at 100° C. overnight, and analyzed using typical characterization technique (PXRD), as illustrated in FIG. 33.

The supernatants were collected and used to quantify the amount of the remaining biomolecule in solution (not adsorbed by the adjuvants), via microBCA protein determination assays. The solutions were filtered with using Syringe Filters with PTFE membranes of 0.2 μm pore size prior to analysis.

As shown in FIG. 33, the PXRD patterns indicated the formation of a crystalline structure with and without BSA.

The amount of remaining BSA, detected in the supernatants, was below 45% of the introduced BSA, indicating an immobilization efficiency >55%.

This study indicates that Al-muconate is suitable for the immobilization of antigens, and in particular BSA.

### Example 26

### Synthesis of Al-Furandicarboxylate MOF

For the synthesis of Al-furandicarboxylate MOF, 2 mL H₂O was added to 324 mg Al(OH)(CH₃COO)₂ and 312 mg of 2,5-furandicarboxylic acid. The mixture was left under stirring at room temperature for 72 h.

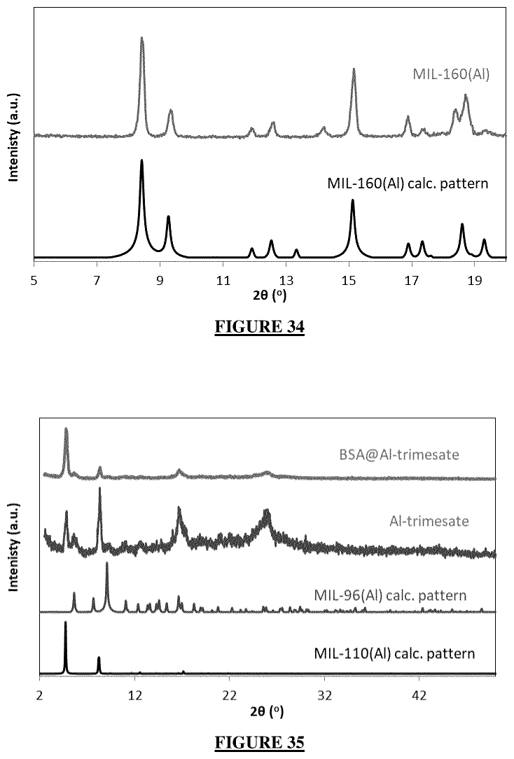

The product was recovered by centrifugation (20 min, 21 200 g), washed 3 times with water, dried at 100° C. overnight and analyzed using typical characterization technique (PXRD), as illustrated in FIG. 34.

The calculated PXRD pattern of MIL-160(Al)_H₂O was obtained from the CCDC; deposition number: 1828694, database identifier: PIBZOS.

The characterizations were in agreements with the formation of hydrated MIL-160.

### Example 27

Biomolecule Immobilization within of Al-Trimesate MOF

For the synthesis of BSA@Al-trimesate, 700 mg Al₂(SO₄)₃·xH₂O (x˜18) were dissolved in 20 mL H₂O in 30 ml vials. Then, was added to the solution 440 mg trimesic acid (1,3,5-benzenetricarboxylic acid) and 256 mg NaOH. A few minutes after, 200 μL BSA (0.15 mg/μL) was added, and the mixture was left under stirring at room temperature for 24 hours, at atmospheric pressure.

A control experiment was performed without the addition of BSA.

The products were recovered by centrifugation (20 min, 21 200 g), washed 3 times with water and dried at 100° C. overnight, and analyzed using typical characterization technique (PXRD), as illustrated in FIG. 35.

The supernatants were collected and used to quantify the amount of the remaining biomolecule in solution (not adsorbed by the adjuvants), via microBCA protein determination assays. The solutions were filtered with using Syringe Filters with PTFE membranes of 0.2 μm pore size prior to analysis.

Calculated PXRD pattern of MIL-110 and MIL-96 were obtained from CCDC; deposition number: 1538658 and 1558833, database identifier: GAWBUE and WEVYEE, respectively.

As shown in FIG. 35, the PXRD patterns indicated the formation of a MIL-110 structure with traces of a MIL-96 structure. The structures were obtained with and without BSA.

The amount of remaining BSA, detected in the supernatants, was below 5% of the introduced BSA, indicating an immobilization efficiency >95%.

This study indicates that Al-trimesate is suitable for the immobilization of antigens, and in particular BSA.

### Example 28

Biomolecule Immobilization within Al-Pyromellitate MOF

For the synthesis of BSA@Al-pyromellitate, 700 mg Al₂(SO₄)₃·xH₂O (x˜18) were dissolved in 10 mL milliQ H₂O. A separate solution, containing 532 mg pyromellitic acid (1,2,4,5-Benzenetetracarboxylic acid) and 384 mg NaOH in 10 mL milliQ H₂O was prepared and added to the metal salt solution. 200 L BSA (0.15 mg/μL) was immediately added to the mixture, and the mixture was left under stirring at room temperature for 24 hours, at atmospheric pressure.

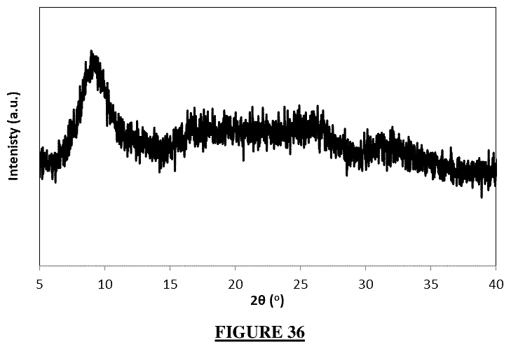

The products were recovered by centrifugation (20 min, 21 200 g), washed 3 times with water and dried at 100° C. overnight, and analyzed using typical characterization techniques (PXRD, FT-IR, TGA), as illustrated in FIG. 36.

The supernatants were collected to quantify the amount of the remaining biomolecule in solution (not adsorbed by the adjuvants), via microBCA protein determination assays.

As shown in FIG. 36, the PXRD patterns indicated the formation of a semi-crystalline structure with BSA.

The amount of remaining BSA, detected in the supernatants, was below 10% of the introduced BSA, indicating an immobilization efficiency >90%.

This study indicates that Al-pyromellitate is suitable for the immobilization of antigens, and in particular BSA.

### Results

Ig and IgG Ab responses were obtained using both Al-fumarate and Alhydrogel® adjuvants. The responses were proportional to the TT and adjuvant concentrations used.

At all tested concentration, Al-fumarate induced a statistically significant stronger Ab response than Alhydrogel®.

In conclusion, it has been shown that antigen is fully immobilized in Al-fumarate according to the invention.

Further, antigen does not influence synthesis and structure of Al-fumarate.

Moreover, Al-fumarate according to the invention has better immobilization capacity than comparative Alhydrogel®. The immobilization with Al-fumarate according to the invention is also more stable than comparative Alhydrogel®.

Al-fumarate according to the invention is stable in the injection media (HEPES, mM pH 7.4) for at least two months.

Al-fumarate according to the invention is partially degraded in vitro in serum/plasma, under concentrated conditions.

Al-fumarate according to the invention resorbs from the injection site.

Al-fumarate according to the invention is suitable for the design of stable vaccine formulation, preserving its immunogenicity for at least to 9 months.

It has been shown that formulations containing TT@Al-fumarate-Surf are stable.

Immobilization of the antigen with Al-MOF, and in particular TT in Al-fumarate, leads to a slower release of the antigen at the injection site than without the MOF.

It has been highlights the absence of acute toxicity, the absence of storage of aluminum in the organism and preserved tissues, with TT@Al-fumarate.

Contrary to the present invention, zinc-based MOF are not suitable for the immobilization of all antigens.

Al-fumarate according to the invention is suitable for the immobilization of inactivated bacteria preserving their morphological aspect, and in particular inactivated E. coli.

Al-fumarate according to the invention is suitable for the immobilization of inactivated bacteria preserving their immunogenic potential and acts as adjuvant leading to an enhanced immune response compared to bare inactivated bacteria and even to the reference Alhydrogel® adjuvant.

Al-fumarate according to the invention is suitable for the immobilization of inactivated viruses, and in particular inactivated poliomyelitis virus from IMOVAX® POLIO vaccine, of glycan, and in particular those from PNEUMOVAX® vaccine, of nucleic acid, and in particular CpG 1018, and of nucleic acid and proteins together, and in particular CpG1018 and Tetanus Toxoid.

Al-muconate, Al-trimesate and Al-pyromellitate according to the invention is suitable for the immobilization of antigens, and in particular BSA.

1. An immunogenic composition containing at least one antigen and at least one adjuvant with:
the adjuvant comprising at least one Metal-Organic Framework, MOF, comprising an inorganic part based on aluminum and an organic part based on at least one polydentate ligand chosen from fumarate, muconate, mesaconate, oxalate, oxaloacetate, succinate, malate, citrate, aconitate, isophthalate, substituted isophthalate, 2,5-thiophenedicarboxylate, 2,5-furandicarboxylate, trimesate, trimellitate and pyromellitate, and
the antigen being immobilized at least within said Metal-Organic Framework.

2. The immunogenic composition according to claim 1, wherein the at least one Metal-Organic Framework is crystallized.

3. The immunogenic composition according claim 1, wherein the at least one Metal-Organic Framework is porous.

4. The immunogenic composition according to claim 1, wherein the organic part of the Metal-Organic Framework based on polydentate ligand comprises at least one fumarate.

5. The immunogenic composition according to claim 1, comprising at least one antigen chosen from proteins, polyosides, lipids, nucleic acids, viruses, bacteria, parasites and mixtures thereof.

6. The immunogenic composition according to claim 1, the adjuvant being resorptive.

7. The immunogenic composition according to claim 1, further comprising at least one antigen that is not immobilized within the Metal-Organic Framework.

8. A Metal-Organic Framework comprising an inorganic part based on aluminum and an organic part based on polydentate ligand chosen from fumarate, muconate, mesaconate, oxalate, oxaloacetate, succinate, malate, citrate, aconitate, isophthalate, substituted isophthalate, 2,5-thiophenedicarboxylate, 2,5-furandicarboxylate, trimesate, trimellitate and pyromellitate, for use to immobilize an antigen, in a vaccine adjuvant, said antigen being immobilized at least within said Metal-Organic Framework.

9. The Metal-Organic Framework according to claim 8, the vaccine adjuvant being resorptive.

10. The Metal-Organic Framework according to claim 8, the organic part based on polydentate ligands comprising at least one fumarate.

11-13. (canceled)

14. A process for preparing an immunogenic composition according to claim 1, comprising at least the step consisting to react at least one aluminum compound with at least one polycarboxylic acid chosen from fumaric acid, muconic acid, mesaconic acid, oxalic acid, oxaloacetic acid, succinic acid, malic acid, citric acid, aconitic acid, isophthalic acid, substituted isophthalic acid, 2,5-thiophenedicarboxylic acid, 2,5-furandicarboxylic acid, trimesic acid, trimellitic acid or pyromellitic acid and/or with at least one polycarboxylate chosen from fumarate, muconate, mesaconate, oxalate, oxaloacetate, succinate, malate, citrate, aconitate, isophthalate, substituted isophthalate, 2,5-thiophenedicarboxylate, 2,5-furandicarboxylate, trimesate, trimellitate and pyromellitate, in the presence of at least one antigen, for forming at least one Al-polycarboxylate Metal-Organic Framework immobilizing said antigen.

15. The process according to claim 14, the aluminum compound being aluminum sulfate.

16. The process according to claim 14, wherein said polycarboxylic acid is at least fumaric acid.

17. The process according to claim 14, wherein the reaction is carried out in an aqueous medium, in particular consisting exclusively of water.

18. The process according to claim 14, wherein the reaction is carried out in the presence of a base.

19. The process according to claim 14, wherein the reaction is carried out at a temperature ranging from 4° C. to 75° C.

20. The process according to claim 14, wherein the molar ratio of the aluminum compound used for the reaction to polycarboxylic acid and/or polycarboxylate varies from 0.001 to 2.5.

21. The process according to claim 14, further comprising a centrifugation step at the end of the reaction, and then optionally a redispersion step.
